(19) United States
(12) Patent Application Publication
(10) Pub. No.: US 2004/0146959 A1
(43) Pub. Date: Jul. 29, 2004

(54) Fluorescent enzyme assay methods and compositions

(76) Inventors: Ronald J. Graham, San Ramon, CA (US); Linda G. Lee, Palo Alto, CA (US); Richard L. Noble, Foster City, CA (US); Hongye Sun, San Mateo, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/659,529

(22) Filed: Sep. 9, 2003

Related U.S. Application Data
(60) Provisional application No. 60/409,178, filed on Sep. 9, 2002.
(60) Provisional application No. 60/486,393, filed on Jul. 10, 2003.

(51) Int. Cl. C12Q 1/48

(52) U.S. Cl. 435/15; 435/21

(57) ABSTRACT

Disclosed are fluorescent compositions and methods for detecting and/or characterizing enzymes and various uses thereof.

### CROSS-REFERENCE TO RELATED APPLICATIONS

This application claims benefit under 35 U.S.C. § 119(e) to application Serial No. 60/409,178, entitled “Fluorescent Enzyme Assay Methods and Composition,” filed Sep. 9, 2002 and application Serial No. 60/486,393, entitled “Fluorescent Enzyme Assay Methods and Compositions,” filed Jul. 10, 2003, the disclosures of which are incorporated herein by reference in their entireties.

### FIELD OF THE INVENTION

The present invention relates to fluorescent compositions and methods for detecting or characterizing enzymes and various uses thereof.

### INTRODUCTION

Enzyme assays are important tools for studying and detecting enzymes for biological and industrial applications. In living organisms, enzymes perform a multitude of tasks, such as synthesis and replication of nucleic acids, modification, and degradation of polypeptides, synthesis of metabolites, and many other functions. Enzymes are also used in industry for many purposes, such as proteases used in laundry detergents, metabolic enzymes to make specialty chemicals such as amino acids and vitamins, and chirally specific enzymes to prepare enantiomerically pure drugs. In medical testing, enzymes are important indicators of the health or disease of human patients.

Although numerous approaches have been developed for assaying enzymes, there is still a great need to find new assay designs that can be used to inexpensively and conveniently detect and characterize a wide variety of enzymes. For example, protein kinases constitute a large class of enzymes that mediate a vast number of fundamental cellular processes. The recent availability of a nearly complete sequence for the human genome has now made possible the identification of many protein kinase candidates that will require years of research to uncover their various metabolic roles (see for example J. C. Venter et al., Science 291:1304-1351 (2001)). Such studies could be significantly facilitated by new assays that are suitable for high throughput screening. However, currently available assay protocols are inconvenient, expensive, or have other deficiencies.

### SUMMARY OF THE INVENTION

In one aspect, the invention provides a method for detecting the phosphorylation activity of one or more protein kinases in a sample. In the method, a mixture is provided comprising a sample and at least one kinase substrate, wherein the kinase substrate comprises (a) a protein kinase recognition moiety containing at least one unphosphorylated residue that is capable of being phosphorylated by a protein kinase, (b) a hydrophobic moiety, and (c) a fluorescent moiety. The mixture is subjected to conditions effective to allow phosphorylation of the unphosphorylated residue when a protein kinase is present in the sample, thereby increasing a fluorescent signal produced by the fluorescent moiety. Detection of an increase in fluorescent signal indicates the presence of protein kinase in the sample.

The protein kinase to be detected can be any protein kinase known in the art. For example, in one embodiment, the protein kinase is a protein kinase A. In another embodiment, the protein kinase is a protein kinase C. In another embodiment, the protein kinase is a protein kinase candidate, and the method is used to confirm and/or characterize the kinase activity of the candidate.

The protein kinase substrate can be designed to be reactive with a particular protein kinase or a group of protein kinases, or it can be designed to determine substrate specificity and/or other catalytic features, such as determining a value for kcat or Km. The unphosphorylated residue in the protein kinase recognition moiety may be any group that is capable of being phosphorylated by a protein kinase. In one embodiment, for example, the residue is a tyrosine residue. In another embodiment, the residue is a serine residue. In yet another embodiment, the residue is a threonine residue.

In addition to having one or more unphosphorylated residues capable of being phosphorylated, the recognition moiety may include additional amino acid residues (or analogs thereof) that facilitate binding specificity, affinity, and/or rate of phosphorylation by the protein kinase to be detected. In some embodiments, the recognition moiety comprises at least 3, 4, 5, 6 or 7 amino acid residues.

The hydrophobic moiety of the substrate is capable of integrating the substrate into a micelle. In one embodiment, the hydrophobic moiety comprises a hydrocarbon moiety comprising from 6 to 30 saturated carbon atoms. Other embodiments are discussed further below. The hydrophobic moiety is preferably chosen to facilitate an increase in fluorescence of the fluorescent moiety upon phosphorylation of the substrate, such that the amplitude of the increase is greater than would be obtained with the same substrate structure lacking the hydrophobic moiety.

The substrate may be designed to have a particular net charge in the unphosphorylated state. In one embodiment, the substrate has a net charge of 0 (a net neutral charge), or about 0, when measured at pH 8, such that addition of a phosphate group yields a product having a net charge of negative 2. In other embodiments, the substrate has a net charge that is different from 0, such as −1, −2, or +1. In one embodiment, the net charge of the substrate is 0 or less. In another embodiment, the net charge is −1 or less.

The fluorescent moiety may be any fluorescent entity that is operative in accordance with the invention. In one embodiment, the fluorescent moiety comprises a fluorescein. In another embodiment, the fluorescent moiety comprises a sulfofluorescein. In another embodiment, the fluorescent moiety comprises a rhodamine. Other fluorescent moieties may also be used.

The protein kinase recognition moiety, hydrophobic moiety, and fluorescent moiety are connected in any way that permits them to perform their respective functions. In one embodiment, the hydrophobic moiety and the fluorescent moiety are linked to each other through the protein kinase recognition moiety. For example, the hydrophobic moiety and the fluorescent moiety can be linked to opposite ends of the part of the substrate that contains the recognition moiety. In another embodiment, the hydrophobic moiety and the recognition moiety are linked to each other through the fluorescent moiety. In another embodiment, a trivalent linker links the hydrophobic moiety, the fluorescent moiety, and the recognition moiety.

The mixture may include a single kinase substrate, or it may include a plurality of different kinase substrates. When the mixture includes a plurality of different kinase substrates, the substrates may differ from one another with respect to any one or more of their protein kinase recognition moieties, hydrophobic moieties and/or fluorescent moieties. As a specific example, the mixture can include two kinase substrates that differ from one another with respect to at least their fluorescent moieties. In one embodiment, the different fluorescent moieties can be selected such that their fluorescence spectra are resolvable from another. For example, the fluorescent moiety on a first kinase substrate may be selected to fluoresce in the green region of the spectrum and the fluorescent moiety on a second kinase substrate selected to fluoresce in the red region of the spectrum. In this embodiment, the kinase substrates can also differ from one another with respect to the specificities of their kinase recognition moieties, permitting the ability to carry out the method in a “multiplexed” fashion, where substrates specific for different kinases or kinase families are correlated with a particular fluorescence signal. When kinase substrates having such spectrally resolvable fluorescent moieties are used, the fluorescent moieties can be selected to have different absorbance or excitation spectra or maxima, or all or a subset may be selected to have similar absorbance or excitation spectra or maxima such that they can be simultaneously excited with a single excitation source.

When a plurality of different kinase substrates are used, although not required for operation, the fluorescent moieties on one or more of the substrates can be selected such that they quench the fluorescence of the fluorescent moieties on one or more of the other substrates when the moieties are in close proximity to one another such as, for example, by collisional quenching, fluorescence resonance energy transfer (FRET) or by another mechanism (or combination of mechanisms). As a specific example, the fluorescent moiety of a first kinase substrate can be selected that has an absorbance spectrum that sufficiently overlaps the emissions spectrum of the fluorescent moiety of a second kinase substrate such that the first fluorescent moiety substantially quenches the fluorescence of the second fluorescent moiety when the two are in close proximity to one another, such as when both kinase substrates are integrated into the same micelle. As another specific example, the fluorescent moieties of two (or more) different kinase substrates may be selected such that they quench the fluorescence of each other when in close proximity thereto.

Although not required for operation, the mixture may optionally include one or more amphipathic quenching molecules capable of quenching the fluorescence of a fluorescent moiety of a kinase substrate when the kinase substrate and the quenching molecule are in close proximity to one another, such as when the kinase substrate and quenching molecule are integrated into the same micelle. Such quenching molecules generally comprise a hydrophobic moiety capable of integrating the quenching molecule into a micelle and a quenching moiety. Specific embodiments of the hydrophobic moiety can include any of the hydrophobic moieties discussed in connection with the kinase substrates.

The quenching moiety can be any moiety capable of quenching the fluorescence of the fluorescent moiety of the kinase substrate. In some embodiments, the quenching moiety can itself be a fluorescent moiety that is capable of quenching the fluorescence of the fluorescent moiety of the kinase substrate when placed in close proximity thereto, such as, for example, by collisional quenching, fluorescence resonance energy transfer (FRET) or by another mechanism (or combination of mechanisms). As a specific example, the quenching moiety can be a fluorescent moiety having an absorbance spectrum that sufficiently overlaps the emissions spectrum of the fluorescent moiety of the kinase substrate such that the quenching moiety substantially quenches the fluorescence of the kinase substrate fluorescent moiety when the quenching moiety and fluorescent moiety of the kinase substrate are in close proximity to one another, such as when the quenching molecule and kinase substrate are integrated into the same micelle. In other embodiments, the quenching moiety is non-fluorescent. The quenching molecule can optionally include a protein kinase recognition moiety.

In another aspect, the invention provides a method for detecting a phosphatase activity of one or more protein phosphatases in a sample. In the method, a mixture is provided comprising a sample and at least one phosphatase substrate, wherein the phosphatase substrate comprises (a) a phosphatase recognition moiety containing at least one phosphorylated residue that is capable of being dephosphorylated (hydrolyzed) by a phosphatase, (b) a hydrophobic moiety, and (c) a fluorescent moiety. The mixture is subjected to conditions effective to allow dephosphorylation of the phosphorylated residue when a phosphatase is present in the sample, thereby increasing a fluorescent signal produced by the fluorescent moiety. Detection of an increase in fluorescent signal in the mixture indicates the presence of a phosphatase in the sample.

The phosphatase to be detected can be any phosphatase known in the art. Also, the phosphatase can be a phosphatase candidate, and the method used to confirm and/or characterize the phosphatase activity of the candidate.

The phosphatase substrate can be designed to be reactive with a particular phosphatase or a group of phosphatases, or it can be designed to determine substrate specificity and other catalytic features, such as determining a value for kcat or Km. The phosphorylated residue in the phosphatase recognition moiety may be any group that is capable of being dephosphorylated by a phosphatase. In one embodiment, for example, the residue is a phosphotyrosine residue. In another embodiment, the residue is a phosphoserine residue. In yet another embodiment, the residue is a phosphothreonine residue.

In addition to having one or more phosphorylated residues capable of being dephosphorylated, the recognition moiety may include additional amino acid residues (or analogs thereof) that facilitate binding specificity, affinity, and/or rate of dephosphorylation by the phosphatase. In some embodiments, the recognition moiety comprises at least 3, 4, 5, 6 or 7 amino acid residues.

The hydrophobic moiety in the substrate is capable of integrating the substrate into a micelle. In one embodiment, the hydrophobic moiety comprises a hydrocarbon moiety comprising from 6 to 30 saturated carbon atoms. Other embodiments are discussed further below. The hydrophobic moiety is preferably chosen to facilitate an increase in fluorescence of the fluorescent moiety upon dephosphorylation of the substrate, such that the amplitude of the increase is greater than would be obtained with the same substrate structure lacking the hydrophobic moiety.

The substrate may be designed to have a particular net charge in the phosphorylated state. In one embodiment, the substrate has a net charge of 0 (a net neutral charge), or about 0, when measured at pH 8, such that removal of a phosphate group yields a product having a net charge of +2. In other embodiments, the substrate has a net charge that is different from 0, such as +1, +2, or −1. In one embodiment, the net charge of the substrate is 0 or greater. In another embodiment, the net charge is +1 or greater.

The fluorescent moiety of the phosphatase substrate may be any fluorescent entity that is operative in accordance with the invention. In one embodiment, the fluorescent moiety comprises a fluorescein. In another embodiment, the fluorescent moiety comprises a sulfofluorescein. In another embodiment, the fluorescent moiety comprises a rhodamine. Other fluorescent moieties may also be used.

The phosphatase recognition moiety, hydrophobic moiety, and fluorescent moiety are connected in any way that permits them to perform their respective functions, in a manner analogous to the design considerations discussed above with respect to the protein kinase substrates.

The mixture may include a single phosphatase substrate, or it may include a plurality of different phosphatase substrates. When the mixture includes a plurality of different phosphatase substrates, the substrates may differ from one another with respect to any one or more of their phosphatase recognition moieties, hydrophobic moieties and/or fluorescent moieties. As a specific example, the mixture can include two phosphatase substrates that differ from one another with respect to at least their fluorescent moieties. In one embodiment, the different fluorescent moieties can be selected such that their fluorescence spectra are resolvable from another. For example, the fluorescent moiety on a first phosphatase substrate may be selected to fluoresce in the green region of the spectrum and the fluorescent moiety on a second phosphatase substrate selected to fluoresce in the red region of the spectrum. In this embodiment, the phosphatase substrates can also differ from one another with respect to the specificities of their phosphatase recognition moieties, permitting the ability to carry out the method in a “multiplexed” fashion, where substrates specific for different phosphatase or phosphatase families are correlated with a particular fluorescence signal. When phosphatase substrates having such spectrally resolvable fluorescent moieties are used, the fluorescent moieties can be selected to have different absorbance or excitation spectra or maxima, or all or a subset may be selected to have similar absorbance or excitation spectra or maxima such that they can be simultaneously excited with a single excitation source.

When a plurality of different phosphatase substrates are used, although not required for operation, the fluorescent moieties on one or more of the substrates can be selected such that they quench the fluorescence of the fluorescent moieties on one or more of the other substrates when the moieties are in close proximity to one another such as, for example, by collisional quenching, fluorescence resonance energy transfer (FRET) or by another mechanism (or combination of mechanisms). As a specific example, the fluorescent moiety of a first phosphatase substrate can be selected that has an absorbance spectrum that sufficiently overlaps the emissions spectrum of the fluorescent moiety of a second phosphatase substrate such that the first fluorescent moiety substantially quenches the fluorescence of the second fluorescent moiety when the two are in close proximity to one another, such as when both phosphatase substrates are integrated into the same micelle. As another specific example, the fluorescent moieties of two (or more) different phosphatase substrates may be selected such that they quench the fluorescence of each other when in close proximity thereto.

Although not required for operation, the mixture may optionally include one or more amphipathic quenching molecules capable of quenching the fluorescence of a fluorescent moiety of a phosphatase substrate when the phosphatase substrate and the quenching molecule are in close proximity to one another, such as when the phosphatase substrate and quenching molecule are integrated into the same micelle. Such quenching molecules generally comprise a hydrophobic moiety capable of integrating the quenching molecule into a micelle and a quenching moiety. Specific embodiments of the hydrophobic moiety can include any of the hydrophobic moieties discussed in connection with the phosphatase substrates.

The quenching moiety can be any moiety capable of quenching the fluorescence of the fluorescent moiety of the phosphatase substrate. In some embodiments, the quenching moiety can itself be a fluorescent moiety that is capable of quenching the fluorescence of the fluorescent moiety of the phosphatase substrate when placed in close proximity thereto, such as, for example, by collisional quenching, fluorescence resonance energy transfer (FRET) or by another mechanism (or combination of mechanisms). As a specific example, the quenching moiety can be a fluorescent moiety having an absorbance spectrum that sufficiently overlaps the emissions spectrum of the fluorescent moiety of the phosphatase substrate such that the quenching moiety substantially quenches the fluorescence of the phosphatase substrate fluorescent moiety when the quenching moiety and fluorescent moiety of the phosphatase substrate are in close proximity to one another, such as when the quenching molecule and phosphatase substrate are integrated into the same micelle. In other embodiments, the quenching moiety is non-fluorescent. The quenching molecule can optionally include a phosphatase recognition moiety.

In a broader aspect, the present invention provides method for detecting or measuring an enzyme activity. In the method, there is provided a mixture comprising a sample and a substrate for the enzyme. The substrate comprises (a) an enzyme recognition moiety that contains a chemical reaction site that is capable of being modified by the enzyme in a manner that changes the net charge of the substrate, (b) a hydrophobic moiety, and (c) a fluorescent moiety. The mixture is subjected to conditions effective to allow the enzyme to modify the chemical reaction site to produce a fluorescently detectable product that contains the modified enzyme recognition moiety, the hydrophobic moiety, and the fluorescent moiety, thereby increasing a fluorescent signal produced by the fluorescent moiety. Detection of an increase in fluorescent signal indicates the presence of the enzyme in the sample.

In one embodiment, the enzyme is a protein kinase. In another embodiment, the enzyme is a protein phosphatase.

In one embodiment, the enzyme recognition moiety comprises a polypeptide segment that contains a group that is chemically altered by the enzyme during the assay to cause an increased fluorescent signal. In some embodiments, the recognition moiety comprises at least 3, 4, 5, 6 or 7 amino acid residues.

The hydrophobic moiety in the substrate is capable of integrating the substrate into a micelle. In one embodiment, the hydrophobic moiety comprises a hydrocarbon moiety comprising from 6 to 30 saturated carbon atoms. Other embodiments are discussed further below. The hydrophobic moiety is preferably chosen to facilitate an increase in fluorescence of the fluorescent moiety upon enzyme reaction with the substrate, such that the amplitude of the increase is greater than would be obtained with the same substrate structure lacking the hydrophobic moiety.

The substrate may be designed to have a particular net charge before reaction with the enzyme. In one embodiment, the substrate has a net charge of 0 (a net neutral charge), or about 0, when measured at pH 8. In other embodiments, the substrate has a net charge that is different from 0, such as −1, −2, or +1 or +2. In one embodiment, the net charge of the substrate is 0 or less. In another embodiment, the net charge is −1 or less. In other embodiments, the net charge of the substrate is 0 or greater or +1 or greater.

In one embodiment, the enzyme reacts with the substrate to add or remove a group that causes a change in the charge of the substrate. For example, reaction of the substrate with the enzyme can cause an increase in the amplitude of the net charge of the substrate, so that the product has a greater negative charge than the substrate or a greater positive charge than the substrate.

The fluorescent moiety may be any fluorescent entity that is operative in accordance with the invention. In one embodiment, the fluorescent moiety comprises a fluorescein. In another embodiment, the fluorescent moiety comprises a sulfofluorescein. In another embodiment, the fluorescent moiety comprises a rhodamine. Other fluorescent moieties may also be used.

The enzyme recognition moiety, hydrophobic moiety, and fluorescent moiety are connected in any way that permits them to perform their respective functions. In one embodiment, the hydrophobic moiety and the fluorescent moiety are linked to each other through the enzyme recognition moiety. For example, the hydrophobic moiety and the fluorescent moiety can be linked to opposite ends of the part of the substrate that contains the recognition moiety. In another embodiment, the hydrophobic moiety and the recognition moiety are linked to each other through the fluorescent moiety. In another embodiment, the hydrophobic moiety, the fluorescent moiety, and the recognition moiety are linked by a trivalent linker.

In another embodiment of the present invention, the action of the enzyme is effective to produce a product that is more fluorescent than the substrate in the reaction mixture, such that the enzyme recognition moiety, hydrophobic moiety, and fluorescent moiety remain present in (are not cleaved from) the product.

The mixture may include a single enzyme substrate or a plurality of enzyme substrates, in a manner analogous to that described above in connection with kinase substrates and phosphatase substrates. The mixture may also include one or more quenching molecules, as discussed above.

The invention also includes fluorescent substrates and compositions and kits containing them, as discussed further herein.

The methods and compositions of the invention may also be used to detect, screen for, and/or characterize substrates, inhibitors, activators, or modulators of enzyme activity, as discussed further herein.

These and other features of the inventions herein will become more apparent from the detailed description.

### BRIEF DESCRIPTION OF THE DRAWINGS

FIG. 1 shows kinetic data (fluorescence versus time) obtained with a protein kinase A in the presence of different concentrations (0.15 μM, 0.3 μM, and 0.6 μM) of a fluorescent protein kinase substrate of the invention.

FIG. 2 shows a double reciprocal plot (1/V plotted as a function of 1/S) generated using the data from FIG. 1.

FIG. 3 shows kinetic data (fluorescence versus time) obtained with a phosphatase in the presence of a fluorescent phosphatase substrate of the invention.

FIG. 4 shows kinetic data in the form of a double reciprocal plot (1/V plotted as a function of 1/S) obtained with a protein kinase A for several concentrations of ATP (50, 10, 3 and 2 μM) in the absence (lowest trace) or presence of the inhibitor staurosporine (5 nM, middle trace) or the PKA-specific peptide inhibitor TYADFIASGRTGRRNAI (20 nM, highest trace).

FIG. 5 shows fluorescence time plots from reaction of protein kinase C-βII with a PKC-βII substrate (compound 8) in the presence of different concentrations of the inhibitor staurosporine (trace A: 0 nM, B: 2 nM, C: 5 nM, and D: 10 nM). The lowest trace (E) was obtained as a control without enzyme.

FIG. 6 shows fluorescence time plots (in triplicate) from reaction of a pp60^{c-src}-related protein tyrosine kinase with a fluorescent substrate (compound 9). Two control reactions were also performed without enzyme (bottom two traces).

FIG. 7A shows raw kinetic data of PKC at ATP=10 uM with 30 seconds reading interval.

FIG. 7B shows the initial velocity data of PKC at ATP=10 uM. Series 1 to 10 represent 0, 0.1 0.5, 1, 2, 5, 10, 20, 50, 100 nM Staurosporine concentration. The linear equations are ordered in the same way.

FIG. 8A shows raw Kinetic data of PKC at ATP=50 uM with 30 seconds reading interval.

FIG. 8B shows the initial velocity data of PKC at ATP=50 uM. Series 1 to 10 represent 0, 0.1 0.5, 1, 2, 5, 10, 20, 50, 100 nM Staurosporine concentration. The linear equations are ordered in the same way.

FIG. 9 shows IC50 for PKC at ATP=10, 50, 100, and 200 uM.

FIG. 10 shows IC50 for PKC at ATP=10, 50, 100, and 200 uM.

### DETAILED DESCRIPTION

II. Definitions

Unless stated otherwise, the following terms and phrases used herein are intended to have the following meanings:

“Detect” and “detection” have their standard meaning, and are intended to encompass detection, measurement, and characterization of a selected enzyme or enzyme activity. For example, enzyme activity may be “detected” in the course of detecting, screening for, or characterizing inhibitors, activators, and modulators of the enzyme activity.

“Micelle” has its standard meaning and is intended to refer to an aggregate formed by amphipathic molecules in water or an aqueous environment such that their polar ends or portions are in contact with the water or aqueous environment and their nonpolar ends or portions are in the interior of the aggregate. A micelle can take any shape or form, including but not limited to, a non-lamellar aggregate that does not enclose a portion of the water or aqueous environment, or a unilamellar or multilamellar vesicle-like aggregate that encloses a portion of the water or aqueous environment, such as, for example, a liposome.

“Quench” has its standard meaning and, in the context of fluorescent signals, is intended to refer to a measurable reduction or decrease in fluorescence intensity at a particular detection wavelength, regardless of the mechanism by which it occurs. By way of illustration are not limitation, a fluorescence signal is quenched when its intensity at a particular detection wavelength is reduced by 25%, 50%, 75%, 80%, 90%, 95% or even more.

Polypeptide sequences are provided with an orientation (left to right) of the N terminus to C terminus, with amino acid residues represented by the standard 3-letter or 1-letter codes (e.g., Stryer, L., Biochemistry, 2^{nd} Ed., W.H. Freeman and Co., San Francisco, Calif., page 16 (1981)).

II. Enzyme Substrate Compositions

The present invention provides enzyme substrates that can be designed to detect any of a large variety of different enzymes. The substrates comprise a hydrophobic moiety capable of integrating the substrate into a micelle. The substrate also contains a fluorescent moiety whose fluorescence increases upon reaction with an enzyme of interest, without requiring a quenching group to suppress the fluorescence of the fluorescent moiety prior to reaction of the substrate with the enzyme. Advantageously, substrates of the invention can be used in a continuous monitoring phase, in real time, to allow the user to rapidly determine whether enzyme activity is present in the sample, and optionally, the amount or specific activity of the enzyme.

By way of illustration, the invention is first discussed below with reference to protein kinases as exemplary enzymes to be detected. In addition to playing important biochemical roles, protein kinases are also useful for illustrating enzymes that cause an increase in net charge of an enzyme substrate by adding a phosphate group to a hydroxyl group to form a phosphorylated substrate. Under basic conditions, phosphorylation of the substrate causes the addition of two negative charges, for a net change in charge of −2. Enzymes that carry out the opposite reaction, protein phosphatases, are also discussed, which cause a net increase in charge of +2 under basic conditions. In either case, the amplitude of the net charge on the enzyme substrate is increased. For example, upon phosphorylation of an enzyme substrate as described above, the amplitude of the net negative charge on the enzyme substrate is increased by −2. On the other hand, upon dephosphorylation of an enzyme substrate by a phosphatase, the amplitude of the net positive charge on the enzyme substrate is increased by +2.

In one embodiment, the invention provides a kinase substrate for detecting or characterizing one or more protein kinases in a sample. In one □xemplary class of compounds, the kinase substrate comprises at least (a) a protein kinase recognition moiety containing at least one unphosphorylated residue that is capable of being phosphorylated by a protein kinase, (b) a hydrophobic moiety capable of integrating the substrate into a micelle, and (c) a fluorescent moiety.

The protein kinase recognition moiety generally includes an amino acid side chain containing a group that is capable of being phosphorylated by a protein kinase. In one embodiment, the phosphorylatable group is a hydroxyl group. Usually, the hydroxyl group is provided as part of a side chain in a tyrosine, serine, or threonine residue, although any other natural or non-natural amino acid side chain or other entity containing a phosphorylatable hydroxyl group can be used. The phosphorylatable group can also be a nitrogen atom, such as the nitrogen atom in the epsilon amino group of lysine, an imidazole nitrogen atom of histidine, or a guanidinium nitrogen atom of arginine. The phosphorylatable group can also be a carboxyl group in an asparate or glutamate residue.

The protein kinase recognition moiety may further comprise a segment, typically a polypeptide segment, that contains one or more subunits or residues (in addition to the phosphorylatable residue) that impart identifying features to the substrate to make it compatible with the substrate specificity of the protein kinase(s) to be detected or characterized.

A wide variety of protein kinases have been characterized over the past several decades, and numerous classes have been identified (see, e.g., S. K. Hanks et al., Science 241:42-52 (1988); B. E. Kemp and R. B. Pearson, Trends Biochem. Sci. 15:342-346 (1990); S. S. Taylor et al., Ann. Rev. Cell Biol. 8:429-462 (1992); Z. Songyang et al., Current Biology 4:973-982 (1994); and Chem. Rev. 101:2209-2600, “Protein Phosphorylation and Signaling” (2001)). Exemplary classes of protein kinases include cAMP-dependent protein kinases (also called the protein kinase A family, A-proteins, or PKA's), cGMP-dependent protein kinases, protein kinase C enzymes (PKC's, including calcium dependent PKC's activated by diacylglycerol), Ca²⁺/calmodulin-dependent protein kinase I or II, protein tyrosine kinases (e.g., PDGF receptor, EGF receptor, and Src), mitogen activated protein (MAP) kinases (e.g., ERK1, KSS1, and MAP kinase type I), cyclin-dependent kinases (CDk's, e.g., Cdk2 and Cdc2), and receptor serine kinases (e.g., TGF-β). Exemplary consensus sequences for various protein kinases are shown in Table 1, below. These various consensus sequences can be used to design protein kinase recognition moieties having desired specificities for particular kinases and/or kinase families.

Protein kinase recognition moieties having desired specifities for particular kinases and/or kinase families can also be designed, for example, using the methods and/or exemplary sequences described in Brinkworth et al., Proc. Natl. Acad. Sci. USA 100(1):74-79 (2003).

TABLE 1

| Symbol | Description | Consensus Sequence^{a} |
|---|---|---|
| PKA | cAMP-dependent | -R-R-X-S/T-Z- |
| PhK | phosphorylase kinase | -R-X-X-S/T-F-F- |
| cdk2 | cyclin-dependent kinase-2 | -S/T-P-X-R/K |
| ERK2 | extracellular-regulated kinase-2 | -P-X-S/T-P- |
| PKC | protein kinase C | KKKKRFSFK^{b} |
| | | XRXXSXRX |
| CaMKI | Ca²⁺/calmodulin-dependent | LRRLSDSNF^{c} |
| | protein kinase I | |
| CaMKII | Ca²⁺/calmodulin-dependent | KKLNRTLTVA^{d} |
| | protein kinase II | |
| c-Src | cellular form of Rous sarcoma | -E-E-I-Y-E/G-X-F- |
| | virus transforming agent | |
| v-Fps | transforming agent of Fujinami | -E-I-Y-E-X-I/V- |
| | sarcoma virus | |
| Csk | C-terminal Src kinase | -I-Y-M-F-F-F- |
| InRK | Insulin receptor kinase | -Y-M-M-M- |
| EGFR | EGF receptor | -E-E-E-Y-F- |

^{a}see, for example, B. E. Kemp and R. B. Pearson, Trends Biochem. Sci. 15: 342-346 (1990); Z. Songyang et al., Current Biology 4: 973-982 (1994); J. A. Adams, Chem Rev. 101: 2272 (2001) and references cited therein; X means any amino acid residue, “/” indicates alternate residues; and Z is a hydrophobic amino acid, such as valine, leucine or isoleucine
^{b}Graff et al., J. Biol. Chem. 266: 14390-14398 (1991)
^{c}Lee et al., Proc. Natl. Acad. Sci. 91: 6413-6417 (1994)
^{d}Stokoe et al., Biochem. J. 296: 843-849 (1993)

Typically, the protein kinase recognition sequence comprises a sequence of L-amino acid residues. However, any of a variety of amino acids with different backbone or sidechain structures can also be used, such as: D-amino acid polypeptides, alkyl backbone moieties joined by thioethers or sulfonyl groups, hydroxy acid esters (equivalent to replacing amide linkages with ester linkages), replacing the alpha carbon with nitrogen to form an aza analog, alkyl backbone moieties joined by carbamate groups, polyethyleneimines (PEIs), and amino aldehydes, which result in polymers composed of secondary amines. A more detailed backbone list includes N-substituted amide (—CON(R)— replaces —CONH— linkages), esters (—CO₂—), keto-methylene (—COCH₂—) methyleneamino (—CH₂NH—), uthioamide (—CSNH—), phosphinate (—PO₂RCH₂—), phosphonamidate and phosphonamidate ester (—PO₂RNH₂), retropeptide (—NHC(O)—), trans-alkene (—CR═CH—), fluoroalkene (e.g.; —CF═CH—), dimethylene (—CH₂CH₂—), thioether (e.g.; —CH₂SCH₂—), hydroxyethylene (—CH(OH)CH₂—), methyleneoxy (—CH₂O—), tetrazole (—CN₄—), retrothioamide (—NHC(S)—), retroreduced (—NHCH₂—), sulfonamido (—SO₂NH—), methylenesulfonamido (—CHRSO₂NH—), retrosulfonamide (—NHS(O₂)—), and peptoids (N-substituted glycines), and backbones with malonate and/or gem-diaminoalkyl subunits, for example, as reviewed by M. D. Fletcher et al., Chem. Rev. 98:763 (1998) and the references cited therein. Peptoid backbones (N-substituted glycines) can also be used (e.g., H. Kessler, Angew. Chem. Int. Ed. Engl. 32:543 (1993); R. N. Zuckermann, Chemtracts-Macromol. Chem. 4:80 (1993); and Simon et al., Proc. Natl. Acad. Sci. 89:9367 (1992).

The recognition moiety may comprise a polypeptide segment containing the group or residue that is to be phosphorylated. In one embodiment, the polypeptide segment has a polypeptide length equal to or less than 30 amino acid residues, 25 residues, 20 residues, 15 residues, 10 residues, or 5 residues. In another embodiment, the polypeptide segment has a polypeptide length in a range of 3 to 30 residues, or 3 to 25 residues, or 3 to 20 residues, or 3 to 15 residues, or 3 to 10 residues, or 3 to 5 residues, or 5 to 30 residues, or 5 to 25 residues, or 5 to 20 residues, or 5 to 15 residues, or 5 to 10 residues, or 10 to 30 residues, or 10 to 25 residues, or 10 to 20 residues, or 10 to 15 residues. In another embodiment, the polypeptide segment contains 3 to 30 amino acid residues, or 3 to 25 residues, or 3 to 20 residues, or 3 to 15 residues, or 3 to 10 residues, or 3 to 5 residues, or 5 to 30 residues, or 5 to 25 residues, or 5 to 20 residues, or 5 to 15 residues, or 5 to 10 residues, or 10 to 30 residues, or 10 to 25 residues, or 10 to 20 residues, or 10 to 15 residues. In another embodiment, the polypeptide segment contains at least 3, 4, 5, 6 or 7 amino acid residues.

The hydrophobic moiety of the substrate is capable of integrating the substrate into a micelle under the assay conditions used to detect the enzyme. The hydrophobic moiety is preferably chosen to facilitate an increase in fluorescence of the fluorescent moiety upon phosphorylation of the substrate, such that the amplitude of the increase is greater than would be obtained with the same substrate structure lacking the hydrophobic moiety.

The exact length, size and/or composition of the hydrophobic moiety can be varied to obtain the desired results. In one embodiment, the hydrophobic moiety comprises a hydrocarbon (consisting of carbon and hydrogen atoms) comprising from 6 to 30 carbon atoms, or from 6 to 25 carbon atoms, or from 6 to 20 carbon atoms, or from 6 to 15 carbon atoms, or from 8 to 30 carbon atoms, or from 8 to 25 carbon atoms, or from 8 to 20 carbon atoms, or from 8 to 15 carbon atoms, or from 12 to 30 carbon atoms, or from 12 to 25 carbon atoms, or from 12 to 20 carbon atoms. The hydrocarbon may be linear, branched, cyclic, or any combination thereof. Exemplary linear hydrocarbon groups that are fully saturated include C6, C7, C8, C9, C10, C11, C12, C13, C14, C15, C16, C17, C18, C19, C20, C22, C24, and C26 n-alkyl chains. In addition, the hydrocarbon may contain a cyclopropyl, cyclobutyl, cyclopentyl, or cyclohexyl group. In one embodiment, the hydrophobic moiety is fully saturated. In another embodiment, the hydrophobic moiety can comprise one or more carbon-carbon double bonds which may be cis or trans, and/or one or more carbon-carbon triple bonds. In some cases, the hydrophobic moiety may have one or more aryl rings or arylalkyl groups, such as 1 or 2 phenyl rings. Optimization testing can be done by making several substrate compounds having different hydrophobic moieties, as illustrated in Example 3 for the compounds in Scheme 3 below.

In another embodiment, the hydrophobic moiety is a nonaromatic moiety that does not have a cyclic aromatic pi electron system. In another embodiment, if the hydrophobic moiety contains one or more unsaturated carbon-carbon bonds, those carbon-carbon bonds are not conjugated. In another embodiment, the structure of the hydrophobic moiety is incapable of interacting with the fluorescent moiety, by a FRET or stacking interaction, to quench fluorescence of the fluorescent moiety. The present invention also encompasses embodiments that involve a combination of any two or more of the foregoing embodiments.

For embodiments in which the hydrophobic moiety is linked to the fluorescent moiety, it will be understood that the hydrophobic moiety is distinct from the fluorescent moiety because the hydrophobic moiety does not include any of the atoms in the fluorescent moiety that are part of the aromatic or conjugated pi-electron system that produces the fluorescent signal. Thus, if a hydrophobic moiety is connected to the 4 position of a xanthene ring, the hydrophobic moiety does not include any of the aromatic ring atoms of the xanthene ring.

While the basis for increased fluorescence may not be certain, it is contemplated that the fluorescent substrates of the invention are capable of forming micelles in the reaction mixture due to the hydrophobic moiety, so that the fluorescent moieties quench each other due to their close proximity and high local concentration. Micelle formation may be evidenced by an increase in light scatter and/or a shift in the absorbance maximum of the fluorescent moiety. In experiments performed in support of the invention, inclusion of a hydrophobic moiety has been found in some cases to cause a large red shift (by about 20 nm) of the absorbance maximum of the fluorescent moiety. However, it is possible that actual formation of micelles by the substrate is not required for operability of the invention.

The fluorescent moiety in the substrate may be any entity that provides a fluorescent signal that can be used to follow enzyme-mediated phosphorylation. Typically, the fluorescent moiety comprises a fluorescent dye that in turn comprises resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, such as xanthenes, rhodamines, fluoresceins, cyanines, phthalocyanines, squaraines, and bodipy dyes.

In one embodiment, the dye comprises a xanthene-type dye, which contains a fused three-ring system of the form:

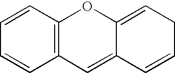

This parent xanthene ring may be unsubstituted (i.e., all substituents are H) or may be substituted with one or more of a variety of the same or different substituents, such as described below.

In one embodiment, the dye contains a parent xanthene ring having the general structure:

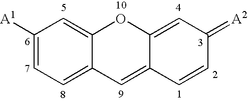

In the parent xanthene ring depicted above, A¹ is OH or NH₂ and A² is O or NH₂⁺. When A¹ is OH and A² is O, the parent xanthene ring is a fluorescein-type xanthene ring. When A¹ is NH₂ and A² is NH₂⁺, the parent xanthene ring is a rhodamine-type xanthene ring. When A¹ is NH₂ and A² is O, the parent xanthene ring is a rhodol-type xanthene ring. In the parent xanthene ring depicted above, one or both nitrogens of A¹ and A² (when present) and/or one or more of the carbon atoms at positions C1, C2, C4, C5, C7, C8 and C9 can be independently substituted with a wide variety of the same or different substituents. In one embodiment, typical substituents include, but are not limited to, —X, —R, —OR, —SR, —NRR, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F or Cl) and each R is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, heteroaryl, 6-26 membered heteroarylalkyl 5-20 membered heteroaryl-heteroaryl, carboxyl, acetyl, sulfonyl, sulfinyl, sulfone, phosphate, or phosphonate. Moreover, the C1 and C2 substituents and/or the C7 and C8 substituents can be taken together to form substituted or unsubstituted buta[1,3]dieno or (C₅-C₂₀) aryleno bridges. Generally, substituents which do not tend to quench the fluorescence of the parent xanthene ring are preferred, but in some embodiments quenching substituents may be desirable. Substituents that tend to quench fluorescence of parent xanthene rings are electron-withdrawing groups, such as —NO₂, —Br, and —I. In one embodiment, C9 is unsubstituted. In another embodiment, C9 is substituted with a phenyl group. In another embodiment, C9 is substituted with a substituent other than phenyl.

When A¹ is NH₂ and/or A² is NH₂⁺, these nitrogens can be included in one or more bridges involving the same nitrogen atom or adjacent carbon atoms, e.g., (C₁-C₁₂) alkyldiyl, (C₁-C₁₂) alkyleno, 2-12 membered heteroalkyldiyl and/or 2-12 membered heteroalkyleno bridges.

Any of the substituents on carbons C1, C2, C4, C5, C7, C8, C9 and/or nitrogen atoms at C3 and/or C6 (when present) can be further substituted with one or more of the same or different substituents. Typical substituents include, but are not limited to —X, —R′, —OR′, —SR′, —NR′R′, —CX₃, —CN, —OCN, —SCN, —NCO, —NCS, —NO, —NO₂, —N₂, —N₃, —NHOH, —S(O)₂O⁻, —S(O)₂OH, —S(O)₂R′, —P(O)(O—)₂, —P(O)(OH)₂, —C(O)R′, —C(O)X, —C(S)R′, —C(S)X, —C(O)OR′, —C(O)O⁻, —C(S)OR′, —C(O)SR′, —C(S)SR′, —C(O)NR′R′, —C(S)NR′R′ and —C(NR)NR′R′, where each X is independently a halogen (preferably —F or —Cl) and each R′ is independently hydrogen, (C₁-C₆) alkyl, 2-6 membered heteroalkyl, (C₅-C₁₄) aryl or heteroaryl, carboxyl, acetyl, sulfonyl, sulfinyl, sulfone, phosphate, or phosphonate.

Exemplary parent xanthene rings include, but are not limited to, rhodamine-type parent xanthene rings and fluorescein-type parent xanthene rings.

In one embodiment, the dye contains a rhodamine-type xanthene dye that includes the following ring system:

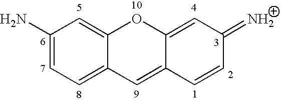

In the rhodamine-type xanthene ring depicted above, one or both nitrogens and/or one or more of the carbons at positions C1, C2, C4, C5, C7 or C8 can be independently substituted with a wide variety of the same or different substituents, as described above for the parent xanthene rings, for example. C9 may be substituted with hydrogen or other substituent, such as an orthocarboxyphenyl or ortho(sulfonic acid)phenyl group. Exemplary rhodamine-type xanthene dyes include, but are not limited to, the xanthene rings of the rhodamine dyes described in U.S. Pat. Nos. 5,936,087, 5,750,409, 5,366,860, 5,231,191, 5,840,999, 5,847,162, and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483. (1992). Also included within the definition of “rhodamine-type xanthene ring” are the extended-conjugation xanthene rings of the extended rhodamine dyes described in U.S. application Ser. No. 09/325,243 filed Jun. 3, 1999.

In another embodiment, the dye comprises a fluorescein-type parent xanthene ring having the structure:

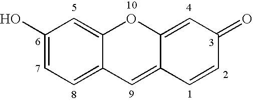

In the fluorescein-type parent xanthene ring depicted above, one or more of the carbons at positions C1, C2, C4, C5, C7, C8 and C9 can be independently substituted with a wide variety of the same or different substituents, as described above for the parent xanthene rings. C9 may be substituted with hydrogen or other substituent, such as an orthocarboxyphenyl or ortho(sulfonic acid)phenyl group. Exemplary fluorescein-type parent xanthene rings include, but are not limited to, the xanthene rings of the fluorescein dyes described in U.S. Pat. Nos. 4,439,356, 4,481,136, 4,933,471 (Lee), U.S. Pat. No. 5,066,580 (Lee), U.S. Pat. Nos. 5,188,934, 5,654,442, and 5,840,999, WO 99/16832, and EP 050684. Also included within the definition of “fluorescein-type parent xanthene ring” are the extended xanthene rings of the fluorescein dyes described in U.S. Pat. Nos. 5,750,409 and 5,066,580.

In another embodiment, the dye comprises a rhodamine dye, which comprises a rhodamine-type xanthene ring in which the C9 carbon atom is substituted with an orthocarboxy phenyl substituent (pendent phenyl group). Such compounds are also referred to herein as orthocarboxyrhodamines. In such rhodamines, the following numbering convention is commonly employed:

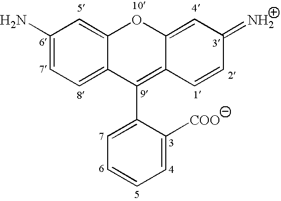

A particularly preferred subset of rhodamine dyes are 4,7,-dichlororhodamines. Typical rhodamine dyes include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dRI 10), tetramethyl rhodamine (TAMRA) and 4,7-dichloro-tetramethylrhodamine (dTARA). Additional rhodamine dyes can be found, for example, in U.S. Pat. No. 5,366,860 (Bergot et al.), U.S. Pat. No. 5,847,162 (Lee et al.), U.S. Pat. No. 6,017,712 (Lee et al.), U.S. Pat. No. 6,025,505 (Lee et al.), U.S. Pat. No. 6,080,852 (Lee et al.), U.S. Pat. No. 5,936,087 (Benson et al.), U.S. Pat. No. 6,111,116 (Benson et al.), U.S. Pat. No. 6,051,719 (Benson et al.), U.S. Pat. Nos. 5,750,409, 5,366,860, 5,231,191, 5,840,999, and 5,847,162, U.S. Pat. No. 6,248,884 (Lam et al.), PCT Publications WO 97/36960 and WO 99/27020, Sauer et al., 1995, J. Fluorescence 5(3):247-261, Arden-Jacob, 1993, Neue Lanwellige Xanthen-Farbstoffe für Fluoresenzsonden und Farbstoff Laser, Verlag Shaker, Germany, and Lee et al., Nucl. Acids Res. 20(10): 2471-2483 (1992), Lee et al., Nucl. Acids Res. 25:2816-2822 (1997), and Rosenblum et al., Nucl. Acids Res. 25:4500-4504 (1997), for example. In one embodiment, the dye comprises a 4,7-dichloro-orthocarboxyrhodamine.

In another embodiment, the dye comprises a fluorescein dye, which comprises a fluorescein-type xanthene ring in which the C9 carbon atom is substituted with an orthocarboxy phenyl substituent (pendent phenyl group). In such fluorescein dyes, the following number convention is commonly employed:

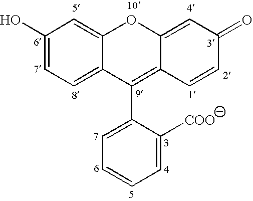

A preferred subset of fluorescein-type dyes are 4,7,-dichlorofluoresceins. Typical fluorescein dyes include, but are not limited to, 5-carboxyfluorescein (5-FAM), 6-carboxyfluorescein (6-FAM). Additional typical fluorescein dyes can be found, for example, in U.S. Pat. Nos. 5,750,409, 5,066,580, 4,439,356, 4,481,136, 4,933,471 (Lee), U.S. Pat. No. 5,066,580 (Lee), U.S. Pat. No. 5,188,934 (Menchen et al.), U.S. Pat. No. 5,654,442 (Menchen et al.), U.S. Pat. No. 6,008,379 (Benson et al.), and U.S. Pat. No. 5,840,999, PCT publication WO 99/16832, and EPO Publication 050684. In one embodiment, the dye comprises a 4,7-dichloro-orthocarboxyfluorescein.

In other embodiments, the dye can be a cyanine, phthalocyanine, squaraine, or bodipy dye, such as described in the following references and references cited therein: U.S. Pat. No. 5,863,727 (Lee et al.), U.S. Pat. No. 5,800,996 (Lee et al.), U.S. Pat. No. 5,945,526 (Lee et al.), U.S. Pat. No. 6,080,868 (Lee et al.), U.S. Pat. No. 5,436,134 (Haugland et al.), U.S. Pat. No. 5,863,753 (Haugland et al.), U.S. Pat. No. 6,005,113. (Wu et al.), and WO 96/04405 (Glazer et al.).

In still other embodiments, the fluorescent moiety can include a network of dyes that can operate cooperatively with one another such as, for example by FRET or another mechanism, to provide large Stoke's shifts. Such dye networks typically include a fluorescence donor moiety and a fluorescence acceptor moiety, and may include moieties that act as both fluorescence acceptors and donors. The fluorescence donor and acceptor moieties can comprise any of the previously described dyes that can act cooperatively with one another. In a specific embodiment, the fluorescent moiety comprises a fluorescence donor moiety which comprises a fluorescein dye and a fluorescence acceptor moiety which comprises a fluorescein or rhodamine dye.

The protein kinase recognition moiety, hydrophobic moiety, and fluorescent moiety are connected in any way that permits them to perform their respective functions. In one embodiment, the hydrophobic moiety and the recognition moiety are linked to each other through the fluorescent moiety. In another embodiment, the hydrophobic moiety and the fluorescent moiety are linked to each other through the protein kinase recognition moiety. For example, the hydrophobic moiety and the fluorescent moiety can be linked to opposite ends of the part of the substrate that contains the recognition moiety. In another embodiment, the hydrophobic moiety, the fluorescent moiety, and the recognition moiety are linked by a trivalent linker.

Scheme 1 below illustrates an embodiment of a substrate in which the hydrophobic moiety and the recognition moiety are linked to each other through the fluorescent moiety. In the illustrated compound (compound 1), a hydrophobic palmitoyl group is linked to the 4 carbon of a fluorescein xanthene ring via an aminomethylbenzoylaminomethyl linker. The protein kinase recognition moiety is linked via an N-terminal phosphoserine residue to a 5-carbonyl group in the pendant phenyl ring of the fluorescein. More generally, the hydrophobic moiety and the recognition moiety may be attached to different sites of the fluorescent moiety (e.g., the 1′, 2′, 4′, 5′, 7′ or 8′ carbon of a xanthene ring, or the 4, 5, 6 or 7 position on the pendant phenyl ring of a rhodamine or fluorescein structure, or the 3′ or 6′ nitrogen atom of a rhodamine), optionally via linkers if appropriate.

Scheme 2 below illustrates an embodiment in which the hydrophobic moiety and the fluorescent moiety can be linked to each other through a protein kinase recognition moiety (when the serine at position 7 is in the unphosphorylated state). The hydrophobic moiety is linked to an N-terminal leucine. The fluorescent moiety is linked to the epsilon amino group of a lysine near the C-terminus of the recognition moiety. Alternatively, one or both of the hydrophobic moiety and the fluorescent moiety can be attached to internal residues within a polypeptide segment. Also, the hydrophobic moiety can be linked to a site in the recognition moiety that is more N-terminal than the site where the fluorescent moiety is attached.

Scheme 3 illustrates yet an embodiment in which the hydrophobic moiety, the fluorescent moiety, and the recognition moiety are linked by a trivalent linker. In the illustrated group of compounds (compounds 3 to 6 and 3P to 6P), a hydrophobic moiety (CH₃(CH₂)ₓ—C(O)— group) is linked to the 2-nitrogen of a 2,3-diaminopropionic acid residue (also referred to herein as an alpha-amino methyl glycine residue, and abbreviated as “Dpr”), and the Dye is linked to the 3-nitrogen of the 2,3-diaminopropionic acid residue. Thus, the 2,3-diaminopropionic acid residue is a trivalent linker. Examples of this kind of substrate are provided in Examples 3-6.

The substrate may be designed to have a particular net charge in the unphosphorylated state. In one embodiment, the substrate has a net charge of 0 (a net neutral charge), or about 0, when measured at pH 8, such that addition of a phosphate group yields a product having a net charge of negative 2. In other embodiments, the substrate has a net charge that is different from 0, such as −1, −2, or +1. In one embodiment, the net charge of the substrate is 0 or less. In another embodiment, the net charge is −1 or less. By increasing the amplitude of the net negative charge of the substrate by −2 due to phosphorylation, a phosphorylated product is formed that is less stable in micellar form than the substrate. Accordingly, the product is more fluorescent that the substrate, so that enzyme activity can be readily detected.

The net charge of the substrate can be established by including an appropriate number of negatively and positively charged groups in the substrate. For example, to establish a net neutral charge (net charge =0), the substrate is designed to contain an equal number of positively and negatively charged groups. Lysine and arginine contain side chains that carry a single positive charge at physiological pH (pH=6 to 8). Aspartate and glutamate contain carboxyl side chains having a single negative charge. A phosphoserine residue carries two negative charges on a phosphate group. The imidazole side chain of histidine has a pK of about 7, so it carries a full positive charge at a pH of about 6 or less. Cysteine has a pK of about 8, so it carries a full negative charge at a pH of about 9 or higher. In addition, the fluorescent may also contain charged groups that should be considered to obtain a particular net charge of the substrate. Guidance regarding the charge state of the substrate is further provided in Section III with respect to Schemes 1 to 4 below.

The substrates of the invention can be readily formed by synthetic methods known in the art. Polypeptides can be prepared by automated synthesizers on a solid support (Perkin J. Am. Chem. Soc. 85:2149-2154 (1963)) by any of the known methods, e.g. Fmoc or BOC (e.g., Atherton, J. Chem. Soc. 538-546 (1981); Fmoc Solid Phase Peptide Synthesis. A Practical Approach, Chan, Weng C. and White, Peter D., eds., Oxford University Press, New York, 2000). Synthetically, polypeptides may be formed by a condensation reaction between the a-carbon carboxyl group of one amino acid and the amino group of another amino acid. Activated amino acids are coupled onto a growing chain of amino acids, with appropriate coupling reagents. Polypeptides can be synthesized with amino acid monomer units where the α-amino group was protected with Fmoc (fluorenylmethoxycarbonyl). Alternatively, the BOC method of peptide synthesis can be practiced to prepare the peptide conjugates of the invention.

Amino acids with reactive side-chains can be further protected with appropriate protecting groups. Amino groups on lysine side-chains to be labelled can be protected with an Mtt protecting group, selectively removable with about 5% trifluoroacetic acid in dichloromethane. A large number of different protecting group strategies can be employed to efficiently prepare polypeptides.

Exemplary solid supports include polyethyleneoxy/polystyrene graft copolymer supports (TentaGel, Rapp Polymere GmbH, Tubingen, Germany) and a low-cross link, high-swelling Merrifield-type polystyrene supports with an acid-cleavable linker (Applied Biosystems), although others can be used as well.

Polypeptides are typically synthesized on commercially available synthesizers at scales ranging from 3 to 50 μmoles. The Fmoc group is removed from the terminus of the peptide chain with a solution of piperidine in dimethylformamide (DMF), typically 30% piperidine, requiring several minutes for deprotection to be completed. The amino acid monomer, coupling agent, and activator are delivered into the synthesis chamber or column, with agitation by vortexing or shaking. Typically, the coupling agent is HBTU, and the activator is 1-hydroxybenzotriazole (HOBt). The coupling solution also may contain diisopropylethylamine or another organic base, to adjust the pH to an optimal level for rapid and efficient coupling.

Peptides may alternatively be prepared on chlorotrityl polystyrene resin by typical solid-phase peptide synthesis methods with a Model 433A Peptide Synthesizer (Applied Biosystems, Foster City, Calif.) and Fmoc/HBTU chemistry (Fields, (1990) Int. J. Peptide Protein Res. 35:161-214). The crude protected peptide on resin may be cleaved with 1% trifluoroacetic acid (TFA) in methylene chloride for about 10 minutes. The filtrate is immediately raised to pH 8 with an organic amine base, e.g. 4-dimethylaminopyridine. After evaporating the volatile reagents, a crude protected peptide is obtained that can be labelled with additional groups.

Following synthesis, the peptide on the solid support (resin) is deprotected and cleaved from the support. Deprotection and cleavage may be performed in any order, depending on the protecting groups, the linkage between the peptide and the support, and the labelling strategy. After cleavage and deprotection, peptides may be desalted by gel filtration, precipitation, or other means, and analyzed. Typical analytical methods useful for the peptides and peptide conjugates of the invention include mass spectroscopy, absorption spectroscopy, HPLC, and Edman degradation sequencing. The peptides and peptide conjugates of the invention may be purified by reverse-phase HPLC, gel filtration, electrophoresis, or dialysis.

Polypeptides may be conjugated, or “labelled”, with a fluorescent dye to provide the fluorescent moiety in the substrate. Typically, a fluorescent dye labelling reagent bears an electrophilic linking moiety which reacts with a nucleophilic group on the polypeptide, e.g. amino terminus, or side-chain nucleophile of an amino acid. Alternatively, the dye may be have a nucleophilic moiety, e.g. amino- or thiol-linking moiety, which reacts with an electrophilic group on the peptide, e.g. NHS of the carboxyl terminus or carboxyl side-chain of an amino acid. The polypeptide may be on a solid support, i.e. synthesis resin, during the labelling reaction. Alternatively, the polypeptide may have been cleaved prior to labelling.

Modification of proteins by labeling with reporter molecules such as fluorescent dyes is a powerful tool in immunology, histochemistry, and cell biology (Means, G. E. and Feeney, R. E. (1971) Chemical Modification of Proteins, Holden-Day, San Francisco, Calif.; Means (1990) Bioconjugate Chem. 1:2; Glazer etal (1975) Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) Chemical Modification of Proteins, In Modern Methods in Protein Chemistry, H. Tschesche, Ed., Walter DeGryter, Berlin and New York; Wong (1991) Chemistry of Protein Conjugation and Cross-linking, CRC Press, Boca Raton, Fla.).

Polypeptides may contain a number of reactive amino acid side chains. Certain amino acid side-chains allow labelling with activated forms of fluorescent dye labelling reagents. Aspartic acid, glutamic acid, lysine, arginine, cysteine, histidine, tyrosine, and other amino acids have reactive functionality for labelling. By appropriate selection of protecting groups, certain reactive functionality on the peptide can be selectively unmasked for reaction with a labelling reagent. Specific reactive moieties can be introduced into the polypeptide by chemical modification of reactive side chains. The reactive side chains may be naturally a part of the protein or are artificially introduced during peptide synthesis or by post-synthesis modification, e.g. by deprotection (Coull, U.S. Pat. No. 6,197,513). They serve as “handles” for attaching a wide variety of molecules, including labels or other proteins. Amines (lysines, α-amino Groups) are the most common reactive groups of proteins, e.g. the aliphatic ε-amine of the amino acid lysine. Lysines are usually present to some extent and are often quite abundant. Lysine amines (pKₐ=9.2) are reasonably good nucleophiles under neutral or basic conditions, e.g. above pH 8.0 (Fasman, G. D. Ed. (1989) Practical Handbook of Biochemistry and Molecular Biology, p13, CRC Press, Boca Raton, Fla.) and therefore react with a variety of reagents to form stable bonds (eq 1).

Protein-NH₂+RX —>Protein-NHR+XH (1)

Other reactive amines that are found in proteins are the α-amino groups of the N-terminal amino acids that are less basic than lysines and are reactive at around pH 7. Sometimes they can be selectively modified in the presence of lysines. There is usually at least one α-amino acid in a protein, and in the case of proteins that have multiple peptide chains or several subunits, there can be more (one for each peptide chain or subunit).

Thiols (sulfhydryls, mercaptans) are another reactive group in the cystine, cysteine, methionine side chains. Cysteine contains a free thiol group, which is more nucleophilic than amines and is generally the most reactive functional group in a protein. It reacts with some of the same modification reagents as do the amines discussed in the previous section and in addition can react with reagents that are not very reactive toward amines. Thiols are reactive at neutral pH, and may be coupled to other molecules selectively in the presence of amines under certain conditions (eq 2).

NH₂-Protein-SH+RX—>NH₂-Protein-SR+XH (2)

Since free thiol groups are relatively reactive, proteins with thiols often exist in their oxidized form as disulfide-linked oligomers or have internally bridged disulfide groups. Reduction of the disulfide bonds with a reagent such as dithiothreitol (DTT) is required to generate the reactive free thiol. In addition to cystine and cysteine, some proteins also have the amino acid methionine, which contains sulfur in a methylthioether form.

Amine-reactive labelling reagents may react with lysines and the α-amino groups of proteins and peptides under both aqueous and nonaqueous conditions. Reactive esters, especially N-hydroxysuccinimide (NHS) esters, are among the most commonly used amine-reactive reagents for modification of polypeptide amine groups. These reagents have high selectivity toward aliphatic amines. Their reaction rates with aromatic amines, alcohols (serine, threonine), phenols (tyrosine), and histidine are relatively low. The aliphatic amide products which are formed are very stable. NHS esters are commercially available with sulfonate groups, with increased water solubility (see Brinkley, 1992, Bioconjugate Chem. 3:2).

Of the many reactions that may be performed at protein amino groups, one useful for labelling purposes is acylation, or reactions that may be considered analogous to acylation. Acylation reactions may be described by the following general scheme:

P—NH₂+X—CO—R→P—NHCO—R+HX

where P is the protein, X is a leaving group and R is the function being introduced, e.g. a fluorescent dye. The active reagent X—CO—R may be produced in situ by the action of an activating agent, such as a carbodiimide, on the free carboxylic acid of the label reagent. Alternatively, stable active esters may be stored as solid reagents. Other amine-reactive labelling reagents, X—O—R, have electrophilic functional groups such as: isothiocyanate (e.g. FITC, fluorescein isothiocyanate), sulphonyl halide and dichlorotriazine. Thiol-reactive labelling reagents include iodoacetyl and maleimido derivatives. Iodoacetyl and maleimido reagents may be used for amine modification also, but a higher pH (>9.0) and longer reaction times are required.

The fluorescent dye label reagents include a reactive linking group, “linking moiety”, at one of the substituent positions for covalent attachment of the dye to a polypeptide. Linking moieties capable of forming a covalent bond are typically electrophilic functional groups capable of reacting with nucleophilic molecules, such as alcohols, alkoxides, amines, hydroxylamines, and thiols. Examples of electrophilic linking moieties include succinimidyl ester, isothiocyanate, sulfonyl chloride, sulfonate ester, silyl halide, 2,6-dichlorotriazinyl, pentafluorophenyl ester, phosphoramidite, maleimide, iodoacetamide, haloacetyl, epoxide, alkylhalide, allyl halide, aldehyde, ketone, acylazide, and anhydride.

The ester N-hydroxysuccinimide (NHS) and the more water-soluble sulphonated form (NHSS), are efficient due to their stability as reagents, convenient reaction times due to their reactivity with protein amino groups (typically 0.5-2 h), and relative ease of synthesis. The NHS ester form of the dye is exemplified by the structure:

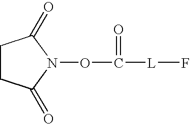

where F is the fluorescent moiety. The linkage L may be a bond or an uncharged linker such as C₁-C₃₀ alkyldiyl, an oxo-alkyl, a terpene, a lipid, a fatty acid, or a steroid. The linker can have functional groups including —C(O)—, —C(O)O—, —O—, —S—, —S—S—, —C(O)NR—, —OC(O)NR, —NRC(O)NR, and —NRC(S)NR; where R is selected from H, C₁-C₆ alkyl and C₅-C₁₄ aryl.

The activated ester, e.g. NHS or HOBt, of the dye may be preformed, isolated, purified, and/or characterized, or it may be formed in situ and reacted with a nucleophilic group of a polypeptide. Typically, a carboxyl substituent of a fluorescent dye is activated by reacting with some combination of: (1) a carbodiimide reagent, e.g. dicyclohexylcarbodiimide, diisopropylcarbodiimide, or a uronium reagent, e.g. TSTU (O -(N-Succinimidyl)-N,N,N′,N′-tetramethyluronium tetrafluoroborate, HBTU (O-benzotriazol-1-yl)-N,N,N′,N′-tetramethyluronium hexafluorophosphate), or HATU (O-(7-azabenzotriazol-1-yl)-N,N,N′,N′-tetramethyluronium hexafluorophosphate); (2) an activator, such as 1-hydroxybenzotriazole (HOBt) or 1-hydroxyazabenotriazole (HOAt); and (3) N-hydroxysuccinimide to give the NHS ester of the dye.

Other activating and coupling reagents include TBTU (2-(1H-benzotriazo-1-yl)-1-1,3,3-tetramethyluronium hexafluorophosphate), TFFH(N,N′,N″,N′″-tetramethyluronium 2-fluoro-hexafluorophosphate), PyBOP (benzotriazole-1-yl-oxy-tris-pyrrolidino-phosphonium hexafluorophosphate, EEDQ (2-ethoxy-1-ethoxycarbonyl-1,2-dihydro-quinoline), DCC (dicyclohexylcarbodiimide); DIPCDI (diisopropylcarbodiimide), MSNT (1-(mesitylene-2-sulfonyl)-3-nitro-1H-1,2,4-triazole, and arylsulfonyl halides, e.g. triisopropylbenzenesulfonyl chloride.

One synthetic route to fluorescent dye labelled polypeptides entails conjugating a fluorescent dye reagent to the N-terminus of a resin-bound peptide before removal of other protecting groups and release of the labeled peptide from the resin. About five equivalents of an amine-reactive fluorophore are usually used per amine of the immobilized peptide. Xanthene fluorophores, including fluoresceins and rhodols are reasonably stable to hydrogen fluoride (HF), as well as to most other acids, after the BOC method of synthesis. These fluorophores are also stable to reagents used for deprotection of peptides synthesized using FMOC chemistry. (Haugland, 1996, Molecular Probes Handbook of Fluorescent Probes and Research Chemicals, Molecular Probes, Inc, Eugene Oreg.).

In another aspect, the invention provides a method for detecting the phosphatase activity of one or more protein phosphatases in a sample. In the method, a mixture is provided comprising a sample and a phosphatase substrate, wherein the phosphatase substrate comprises (a) a phosphatase recognition moiety containing at least one phosphorylated residue that is capable of being dephosphorylated (hydrolyzed) by a phosphatase, (b) a hydrophobic moiety capable of integrating the substrate into a micelle, and (c) a fluorescent moiety. The mixture is subjected to conditions effective to allow dephosphorylation of the phosphorylated residue when a phosphatase is present in the sample, thereby increasing a fluorescent signal produced by the fluorescent moiety. Detection of an increase in fluorescent signal in the mixture indicates the presence of a phosphatase in the sample.

The phosphatase to be detected can be any phosphatase known in the art. Also, the phosphatase can be a phosphatase candidate, and the method is used to confirm and/or characterize the kinase activity of the candidate.

A wide variety of protein phosphatases have been identified (e.g., see P. Cohen, Ann. Rev. Biochem. 58:453-508 (1989), Molecular Biology of the Cell, 3rd edition, Alberts et al., eds., Garland Publishing, NY (1994), and Chem. Rev. 101:2209-2600, “Protein Phosphorylation and Signaling” (2001)). Serine/threonine protein phosphatases represent a large class of enzymes that reverse the action of protein kinase A enzymes, for example. The serine/threonine protein phosphatases have been divided among four groups designated I, IIA, IIB, and IIC. Protein tyrosine kinases are also an important class of phosphatases, and histidine, lysine, arginine, and aspartate phosphatases are also known (e.g., see P. J. Kennelly, Chem Rev. 101:2304-2305 (2001) and references cited therein). In some cases, phosphatases are highly specific for only one or a few proteins, but in other cases, phosphatases are relatively non-specific and can act on a large range of protein targets. Accordingly, the phosphatase substrates of the present invention can be designed to detect particular phosphatases by suitable selection of the phosphatase recognition moiety. Examples of peptide sequences that can be dephosphorylated by phosphatase activity are described in P. J. Kennelly, Chem. Rev. 101:2291-2312 (2001).

The phosphatase substrate can be designed to be reactive with a particular phosphatase or a group of phosphatases, or it can be designed to determine substrate specificity and other catalytic features, such as determining a value for kcat or Km. The phosphorylated residue in the phosphatase recognition moiety may be any group that is capable of being dephosphorylated by a phosphatase. In one embodiment, the residue is a phosphotyrosine residue. In another embodiment, the residue is a phosphoserine residue. In yet another embodiment, the residue is a phosphothreonine residue.

In addition to having one or more phosphorylated residues capable of being dephosphorylated, the recognition moiety may include additional amino acid residues (or analogs thereof) that facilitate binding specificity, affinity, and/or rate of dephosphorylation by the phosphatase.

The recognition moiety may comprise a polypeptide segment containing the group or residue that is to be dephosphorylated. In one embodiment, the polypeptide segment has a polypeptide length equal to or less than 30 amino acid residues, 25 residues, 20 residues, 15 residues, 10 residues, or 5 residues. In another embodiment, the polypeptide segment has a polypeptide length in a range of 3 to 30 residues, or 3 to 25 residues, or 3 to 20 residues, or 3 to 15 residues, or 3 to 10 residues, or 3 to 5 residues, or 5 to 30 residues, or 5 to 25 residues, or 5 to 20 residues, or 5 to 15 residues, or 5 to 10 residues, or 10 to 30 residues, or 10 to 25 residues, or 10 to 20 residues, or 10 to 15 residues. In another embodiment, the polypeptide segment contains 3 to 30 amino acid residues, or 3 to 25 residues, or 3 to 20 residues, or 3 to 15 residues, or 3 to 10 residues, or 3 to 5 residues, or 5 to 30 residues, or 5 to 25 residues, or 5 to 20 residues, or 5 to 15 residues, or 5 to 10 residues, or 10 to 30 residues, or 10 to 25 residues, or 10 to 20 residues, or 10 to 15 residues. In another embodiment, the polypeptide segment contains at least 3, 4, 5, 6 or 7 amino acid residues.

The hydrophobic moiety in the substrate is capable of integrating the substrate into a micelle. The hydrophobic moiety may have the same features as described above with respect to the hydrophobic moiety for the protein kinase substrates above. The hydrophobic moiety is preferably chosen to facilitate an increase in fluorescence of the fluorescent moiety upon dephosphorylation of the substrate, such that the amplitude of the increase is greater than would be obtained with the same substrate structure lacking the hydrophobic moiety.

The substrate may be designed to have a particular net charge in the phosphorylated state. In one embodiment, the substrate has a net charge of 0 (a net neutral charge), or about 0, when measured at pH 8, such that removal of a phosphate group yields a product having a net charge of +2. In other embodiments, the substrate has a net charge that is different from 0, such as +1, +2, or −1. In one embodiment, the net charge of the substrate is 0 or greater. In another embodiment, the net charge is +1 or greater.

The fluorescent moiety of the phosphatase substrate may be any fluorescent entity that is operative in accordance with the invention. In one embodiment, the fluorescent moiety comprises a fluorescein. In another embodiment, the fluorescent moiety comprises a sulfofluorescein. In another embodiment, the fluorescent moiety comprises a rhodamine. Other fluorescent moieties may also be used, of the same type discussed above with respect to protein kinase substrates.

The phosphatase recognition moiety, hydrophobic moiety, and fluorescent moiety are connected in any way that permits them to perform their respective functions, in a manner analogous to the design considerations discussed herein with respect to protein kinase substrates.

More generally, substrates for detecting an enzyme, such as a protein kinase, phosphatase, or other enzyme, may be designed to have any of the following features, including any combinations thereof. In one embodiment, the fluorescence of the product of the enzyme reaction is at least 2 times, at least 3 times, at least 4 times, or at least 5 times the fluorescence of the substrate, on a mole:mole basis. In another embodiment, the substrate has a molecular weight of less than 5000 daltons, or less than 4000 daltons, or less than 3000 daltons, or less than 2000 daltons. In another embodiment, the substrate excludes (does not comprise) structures in which the fluorescent moiety is bound to an apoenzyme or apoprotein.

III. Methods

The sample to be tested may be any suitable sample selected by the user. The sample may be naturally occurring or man-made. For example, the sample may be a blood sample, tissue sample, cell sample, buccal sample, skin sample, urine sample, water sample, or soil sample. The sample can be from a living organism, such as a eukaryote, prokaryote, mammal, human, yeast, or bacterium. The sample may be processed prior to contact with a substrate of the invention by any method known in the art. For example, the sample may be subjected to a precipitation step, column chromatography step, heat step, etc. In some cases, the sample is a purified or synthetically prepared enzyme that is used to screen for or characterize an enzyme substrate, inhibitor, activator, or modulator.

If the sample contains both a kinase and a phosphatase, so that the activity of one may interfere with the activity of the other, then an inactivating agent (e.g., an active site directed an irreversible inhibitor) can be added to the sample to inactivate whichever activity is not desired.

The reaction mixture typically includes a buffer, such as a buffer described in the “Biological Buffers” section of the 2000-2001 Sigma Catalog. Exemplary buffers include MES, MOPS, HEPES, Tris (Trizma), bicine, TAPS, CAPS, and the like. The buffer is present in an amount sufficient to generate and maintain a desired pH. The pH of the reaction mixture is selected according to the pH dependency of the activity of the enzyme to be detected. For example, the pH can be from 2 to 12, from 4 to 11, or from 6 to 10. The reaction mixture also contains any necessary cofactors and/or cosubstrates for the enzyme (e.g., ATP for a protein kinase, Ca²⁺ ion for a calcium dependent kinase, and cAMP for a protein kinase A). Additional mixture components are discussed in Section IV below. In one embodiment, the reaction mixture does not contain detergent or is substantially free from detergents.

In some embodiments, it may be desirable to keep the ionic strength as low as reasonably possible to help avoid masking charged groups in the reaction product, so that micelle formation of product molecules remains disfavored and destabilized. For example, high salt concentration (e.g., 1 M NaCl) may be inappropriate. In addition, it may be desirable to avoid high concentrations of certain other components in the reaction mixture that can also adversely affect the fluorescence properties of the product. Guidance regarding the effects of ionic species, such as metal ions, can be found in Surfactants and Interfacial Phenomena, 2nd Ed., M. J. Rosen, John Wiley & Sons, New York (1989), particularly chapter 3. For example, Mg²⁺ ion at a concentration of 1 mM is useful in the Examples provided below, but higher concentrations may give poorer results.

In practicing certain aspects of the invention, an enzyme substrate of the invention is mixed with a sample containing an enzyme that is to be detected or that is being used to screen for, detect or characterize a compound for substrate, inhibitor, activator, or modulator activity. Reaction of the enzyme with the substrate causes an increase (to a more charged species) in the absolute amplitude of the net charge of the substrate, such that the fluorescence of the reacted substrate is greater than the fluorescence of the unreacted substrate. In one embodiment, the substrate has a net charge of zero (neutral net charge), and reaction of the substrate with the enzyme makes the substrate either (1) net negatively charged by (1A) adding or generating a new negatively charged group on the substrate, or (1B) removing or blocking a positively charged group on the substrate; or (2) net positively charged, by (2A) adding or generating a new positively charged group on the substrate, or (2B) removing or blocking a negatively charged group on the substrate. If the substrate has a net charge that is positive or negative, then the enzyme acts on the substrate to change the net charge to be more negative or less negative, provided that the product is more fluorescent than the substrate in the reaction mixture so that enzyme activity can be detected.

For example, reaction (1A) can be accomplished by adding a phosphate group to a hydroxyl group on the substrate (changing a neutrally charged group to a group having a charge of −2, e.g., using a protein kinase), by cleaving a carboxylic ester or amide to produce a carboxyl group (changing a neutrally charged group to a group having a charge of −1, e.g., using an esterase or amidase). Reaction (1B) can be accomplished by reacting an amino or hydrazine group in the substrate with an acetylating enzyme to produce a neutral acetyl ester group, with an N-oxidase enzyme to produce a neutral N-oxide, with an ammonia lyase to remove ammonia, or with an oxidase that causes oxidative deamination, for example. Reaction (2A) can be accomplished, for example, by treating an amide group in the substrate with an amidase to generate a positively charged amino group in the substrate. Reaction (2B) can be accomplished using a decarboxylase enzyme to remove a carboxylic acid or by reacting a carboxyl group with a methyl transferase to form a carboxylic ester, for example. A variety of enzymes capable of performing such transformations are known in the literature (e.g., see C. Walsh, Enzymatic Reaction Mechanisms, WH Freeman and Co., New York, (1979), the Worthington Product Catalog (Worthington Enzymes), Sigma Life Sciences Catalog, and the product catalogs of other commercial enzyme suppliers).

In another embodiment, the enzyme substrate has a net negative charge, such as −1, −2, −3, −4, or greater, prior to reaction with the enzyme, but the fluorescence of unreacted substrate is sufficiently low so that increasing the net negative charge of the substrate by reaction with the enzyme causes a detectable increase in fluorescence.

Alternatively, in other embodiments the enzyme substrate may have a net positive charge of +1, +2, +3, +4 or greater, prior to reaction with the enzyme, but fluorescence of unreacted substrate is sufficiently low so that increasing the net positive charge of the substrate by reaction with the enzyme causes a detectable increase in fluorescence.

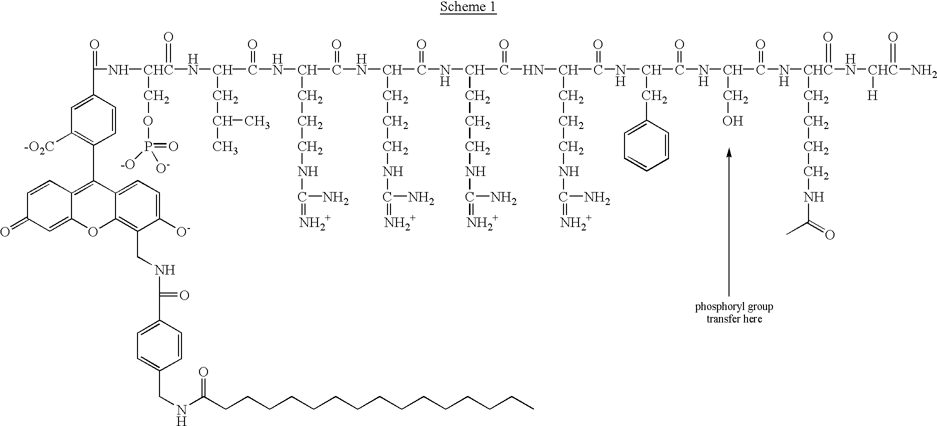

Scheme 1 illustrates an exemplary substrate (compound 1) that can be used to detect protein kinase A. The structure of compound 1 can be represented as X-L-Dye-Ser(OPO₃²⁻)LeuArgArgArgArgPheSerLys(ε-N-Ac)Gly(NH₂), wherein X is a C-16 fatty acid acyl group (palmitoyl), L is a linker (para-NHCH₂C₆H₄C(═O)NHCH₂) that links X to Dye, Dye is a fluorescent moiety (in this case, fluorescein), ε-N—Ac is an acetyl group, Ser, Leu, Arg, Phe, Ser, Lys, and Gly are standard 3-letter codes for serine, leucine, arginine, phenylalanine, lysine, and glycine, respectively, and NH2 indicates that the carboxyl group of the C-terminal glycine is an amide. An exemplary synthesis of compound 1 is described in Example 1A.

As can be seen, compound 1 contains a phenolate anion and a carboxyl anion in the Dye moiety, and a phosphate group in the N-terminal serine residue having two additional negative charges, for a total negative charge of −4. This is offset by the guanidinium groups in the four arginine residues, for a total of four positive charges. Thus, the net charge of the compound is about 0 at pH 8.

Compound 1 further includes a protein kinase recognition moiety in the form of a polypeptide containing an amino acid sequence that is recognized by protein kinase A. The recognition moiety also contains an unphosphorylated serine that is capable of being phosphorylated by the kinase. Upon phosphorylation, the net charge of the substrate is changed from neutral to −2, thereby causing an increase in fluorescence.

While the basis for increased fluorescence is not certain, and the inventors do not wish to be bound to a particular theory, it is contemplated that the fluorescent substrates of the invention are capable of forming micelles in the reaction mixture due to the hydrophobic moiety, so that the fluorescent moieties quench each other due to their close proximity. Micelle formation can be particularly favored when the substrate is neutrally charged or has a small negative or small positive net charge, so that micelle formation is not prevented by mutual charge repulsion. The putative micelles may be in equilibrium with monomolecular, unassociated species in solution, but the micellar form is the predominant form. The product of the enzyme reaction, however, has an increased net charge (total net negative or total net positive) such that micellar formation by the product is disfavored. The free product fluoresces brightly since it remains relatively free from other fluorescent substrate molecules in the solution.

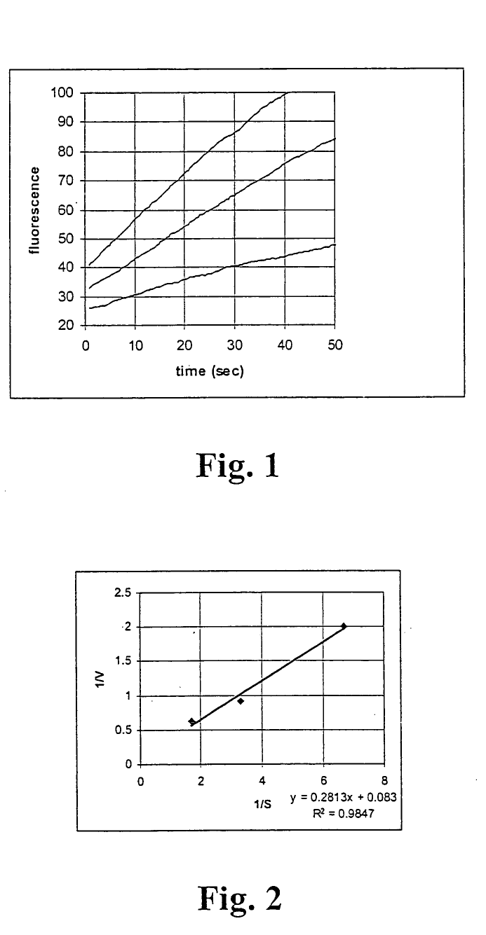

FIG. 1 shows kinetic data obtained with compound 1 in the presence of protein kinase A, using the procedure described in Example 1B. The fluorescence signal of reaction mixtures containing three different concentrations of compound 1 (0.15, 0.30, and 0.60 μM) were monitored over time in the presence of a constant amount of enzyme. As can be seen, the rate of increase in fluorescence was proportional to the amount of compound in the reaction mixture. Furthermore, the data showed a significant increase in fluorescent signal with low noise, so that the signal-to-noise ratio was high. A double reciprocal plot (see FIG. 2) of the initial velocities yielded a Km value of 0.3 μM, consistent with phosphorylation of the substrate by the enzyme.

These results demonstrate that by causing an increased amplitude of net negative charge, by converting a substrate having a net 0 charge to a product having a net negative charge of −2, a significant increase in fluorescence can be generated to detect enzyme activity.

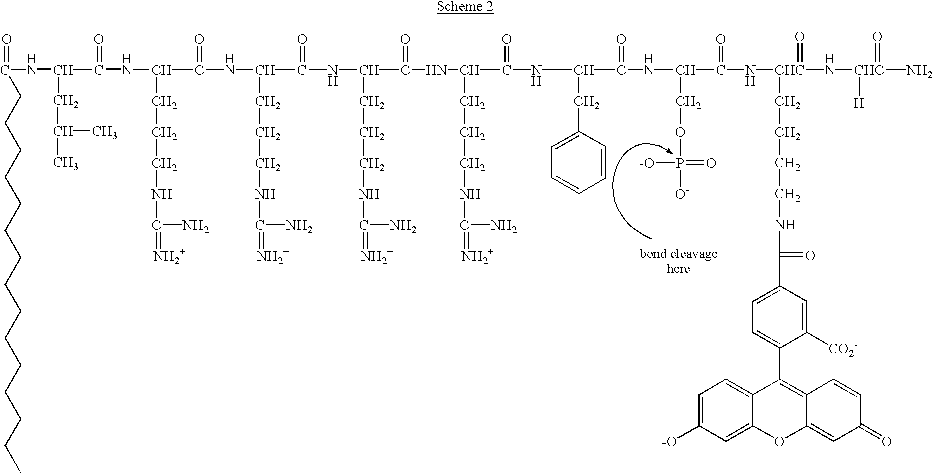

Scheme 2 shows an exemplary substrate (compound 2) for detecting alkaline phosphatase activity. The compound can be represented as X-LeuArgArgArgArgPheSer (OPO₃²⁻)Lys(ε-N-Dye)Gly-NH₂, wherein X is a C-16 fatty acid acyl group (palmitoyl), Dye is a fluorescent moiety (fluorescein) that is linked to the epsilon amino group of a lysine residue, and NH2 indicates that the carboxyl group of the C-terminal glycine is an amide. An exemplary synthesis of compound 2 is described in Example 2A. In this structure, the hydrophobic X group is linked directly by an amide bond to the N-terminal amino group of the polypeptide segment, without using additional linker atoms. However, it will be appreciated that a linker containing one or more linking chain atoms could also be included if desired.

Prior to reaction with phosphatase, the substrate contains a total of four positive charges that are provided by four arginine side chains, and four negative charges which are provided by two negative charges in the fluorescein Dye moiety (a phenolate anion and a carboxyl anion), and two additional negative charges in a phosphate group, for a total net charge of about 0 at pH 8. Upon hydrolysis of the phosphate group from the phosphorylated serine residue adjacent to the phenylalanine residues, the resulting product has a net positive charge of +2, due to loss of the two negative charges on the phosphate group. Accordingly, the product is expected to fluoresce more brightly than the unreacted form, due to micelle instability.

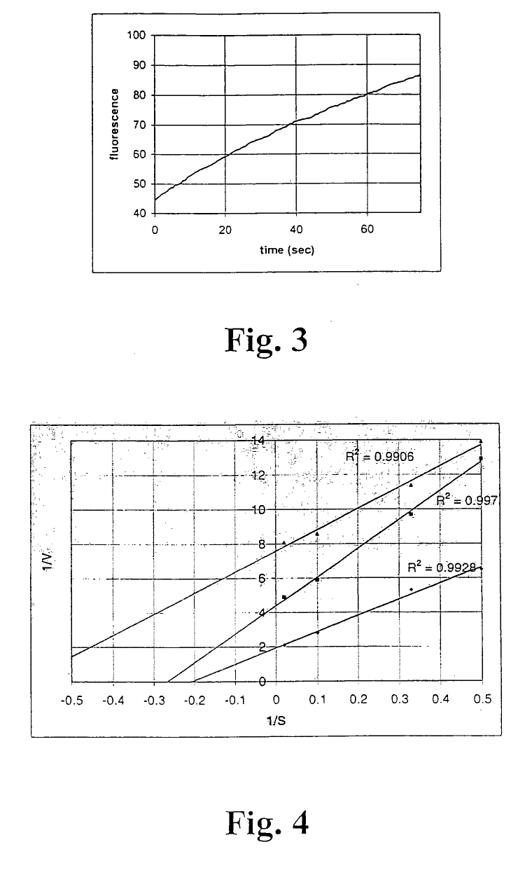

FIG. 3 shows kinetic data from compound 2 in the presence of an alkaline phosphatase according to the procedure described in Example 2B. As can be seen, reaction with compound 2 caused an immediate and significant increase in fluorescence over time with high signal-to-noise ratio. These results demonstrate that by causing an increased amplitude of net charge, in this case by converting a substrate having a net 0 charge to a product having a net positive charge of +2, a significant increase in fluorescence can be generated to detect enzyme activity.

One difference between the structures of compounds 1 and 2 is that the hydrophobic moiety and the fluorescent moiety in compound 2 are located at opposite ends of a polypeptide scaffold, whereas the hydrophobic moiety and the fluorescent moiety in compound 1 are relatively close together at the same end of a polypeptide scaffold. As can be seen from the data shown in FIGS. 1 to 3, both designs are suitable for assaying enzymes in accordance with the invention.

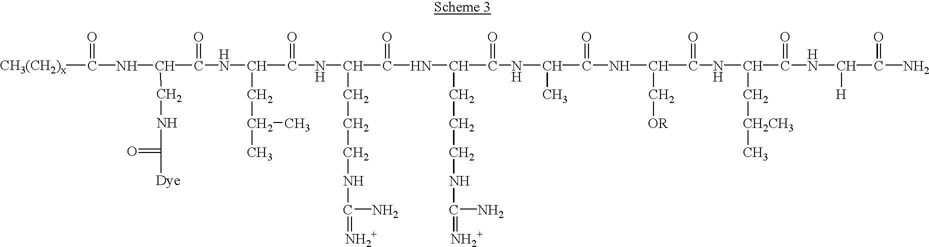

TABLE 1

| Compound | Variables |
|---|---|
| 3 | x = 0, R = H |
| 3P | x = 0, R = PO₃²⁻ |
| 4 | x = 7, R = H |
| 4P | x = 7, R = PO₃²⁻ |
| 5 | x = 10, R = H |
| 5P | x = 10, R = PO₃²⁻ |
| 6 | x = 14, R = H |
| 6P | x = 14, R = PO₃²⁻ |

Yet another design for enzyme substrates in accordance with the invention is illustrated in Scheme 3 (see compounds 3 to 6 and 3P to 6P). Scheme 3 shows a group of compounds having different length alkyl acyl groups (X), as possible substrates for detecting a protein kinase A by fluorescence detection. The general structure of these substrates can be represented by X-Y(Dye)-LeuArgArgAlaSer(OR)LeuGly-NH₂, wherein X is a fatty acid acyl group of the form CH₃(CH₂)ₙC(═O)—, with x as defined in Table 1, Y is 2-aminomethylglycine, Dye is a 4,7-dichlorofluorescein dye attached to the 2-amino group of Y by a 5-carbonyl linkage to the pendant phenyl ring of the dye, R is H or PO₃²⁻ (see table 1), and NH2 indicates that the carboxyl group of the C-terminal glycine is an amide. An exemplary synthesis of compound 3 is shown in Example 3A.

Each of these substrates contains a total of two positive charges from two arginine side chains, and two negative charges from the fluorescein Dye moiety (a phenolate anion and a carboxyl anion), for a total net charge of about 0 at pH 8. The substrate contains an unphosphorylated serine residue that is capable of being phosphorylated by the kinase. Upon phosphorylation, the net charge of the substrate is changed from neutral to −2.

To be effective, not only should a substrate react with the enzyme to form the desired modified product, but also the product should be more fluorescent than the substrate, so that a detectable increase in fluorescence can be observed. Generally, a greater change in fluorescence provides greater assay sensitivity, provided that an adequately low signal-to-noise ratio is achieved. Therefore, it may be desirable to test multiple substrate variants to find a substrate having the most suitable fluorescence properties.

Example 3 describes a study in which several compounds having the structure shown in Scheme 3 above were prepared in phosphorylated and unphosphorylated forms.

25. The substrate structures differed in the lengths of their hydrocarbon “tails” in the hydrophobic moiety (X), with chain lengths of 1, 8, 11 and 15 saturated carbon atoms. Results are shown in Table 2.

TABLE 2

| | Hydrocarbon | | | Fluorescence |
|---|---|---|---|---|
| Compound | Tail Length | F (unphos.)¹ | F (phos.)¹ | Ratio (approx)² |
| 3, 3P | 1 | 1680 | 1930 | 1 |
| 4, 4P | 8 | 575 | 1370 | 2 |
| 5, 5P | 11 | 45 | 431 | 10 |
| 6, 6P | 15 | 3 | 20 | 7 |

¹Fluorescence measurements in arbitrary units for unphosphorylated (unphos.) or phosphorylated (phos.) form of the substrate.
²Rounded value of F(phos)/F(unphos)

As can be seen in Table 2, for compounds 3 and 3P, virtually no difference in fluorescence is observed between the unphosphorylated (unphos.) and the phosphorylated form. This indicates that an acetyl group is too small to favor micelle formation for unphosphorylated substrate. However, significant differences in fluorescence are observed for the longer X groups. The dodecanoyl group (compounds 5, 5P) appears to provide the greatest increase upon phosphorylation (an increase of about 900%), but the tetradecanoyl group (compounds 6, 6P) is also very effective, showing an increase of about 600%). The fluorescence observed for the nonanoyl group (compounds 4, 4P) indicates that this substrate might also be useful. The results demonstrate that the presence of a hydrophobic moiety capable of integrating the substrate into a micelle is effective to cause quenching of fluorescence of unphosphorylated substrate, apparently due to predominance of the self-quenching micellar form, whereas the equilibrium between micellar and free forms of the phosphorylated substrate is shifted in favor of the free form, so that less signal from the phosphorylated substrate is self-quenched.

Table 2 also shows that the amplitude of the fluorescent signals of both forms of each compound decreased with increasing length of the hydrophobic moiety. A possible explanation is that longer hydrophobic chains may cause an increasing proportion of the phosphorylated product to form micelles, so that some of the fluorescent signal of the product is suppressed due to self-quenching. However, if the equilibrium constant between free and micellar forms of the product is greater than the corresponding equilibrium constant for the unphosphorylated substrate, then enzyme-catalyzed phosphorylation can generate an observable increase in fluorescence. For example, compound 5 has been found to be an efficient substrate for E. coli protein kinase A based on fluorescence detection (data not shown).

Another embodiment for enzyme substrates in accordance with the invention includes substrates wherein the hydrophobic moiety may be substituted by at least one halogen atom (e.g. fluorine). Examples of such enzyme substrates are shown in Scheme 4 and Scheme 5. Scheme 4 shows an exemplary substrate (compound 7) for detecting protein kinase A activity. The compound can be represented as X-Lys(ε-N-Dye)LeuArg-ArgAlaSerLeuGly-NH₂, wherein X is a n-(1H, 1H, 2H, 2H perfluorodecyl-1-thiol-2-acetyl group, Dye is a fluorescent moiety (5-carboxysulfofluorescein) that is linked to the epsilon amino group of a lysine residue, and NH2 indicates that the carboxyl group of the C-terminal glycine is an amide. An exemplary synthesis of compound 7 is described in Example 4A. In this structure, the hydrophobic X group is linked by a thiol-2-acetyl group to the N-terminal amino group of the polypeptide segment. However, it will be appreciated that alternative linkers could also be included if desired.

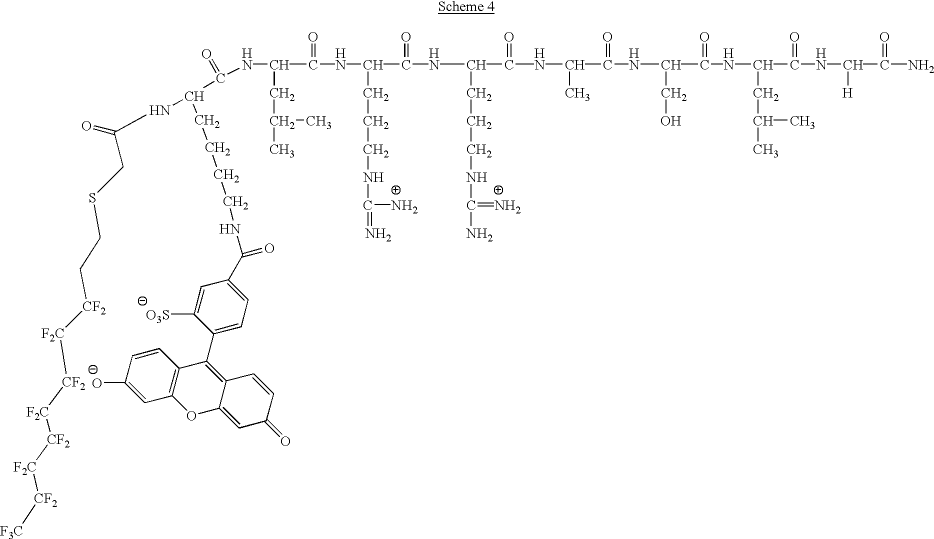

As can be seen, compound 7 contains a phenolate anion and a sulfonate anion in the Dye moiety for a total negative charge of −2. This negative charge in the dye is offset by the two positively charged guanidinium groups in the two arginine residues, for a total of two positive charges. Thus, the net charge of compound 7 is about 0 at pH 8.

Compound 7 further includes a protein kinase recognition moiety in the form of a polypeptide containing an amino acid sequence that is recognized by protein kinase A. The recognition moiety also contains an unphosphorylated serine that is capable of being phosphorylated by the kinase. Upon phosphorylation, the net charge of the substrate is changed from neutral to −2, thereby causing an increase in fluorescence. Fluorescence data for compound 7 can be found in Example 4B.

Scheme 5 shows another exemplary substrate (compound 8) for detecting protein kinase A activity. The compound can be represented as Dye-Lys(ε-N—X)LeuArg-ArgAlaSerLeuGly-NH₂, wherein X is a N-perfluorooctanoylproline that is linked to the epsilon amino group of a lysine residue, Dye is a fluorescent moiety (5-carboxy-2′,7′-dipyridyl-sulfofluorescein), and NH2 indicates that the carboxyl group of the C-terminal glycine is an amide. An exemplary synthesis of compound 8 is described in Example 5A. In this structure, the hydrophobic X group is linked by a proline to the epsilon amino group of a lysine residue. It will be appreciated that alternative linkers may also be included if desired. Furthermore, the fluorescent dye is linked directly by an amide bond to the N-terminal amino group of the polypeptide segment, without using additional linker atoms. However, it will be appreciated that a linker containing one or more linking chain atoms could also be included if desired.

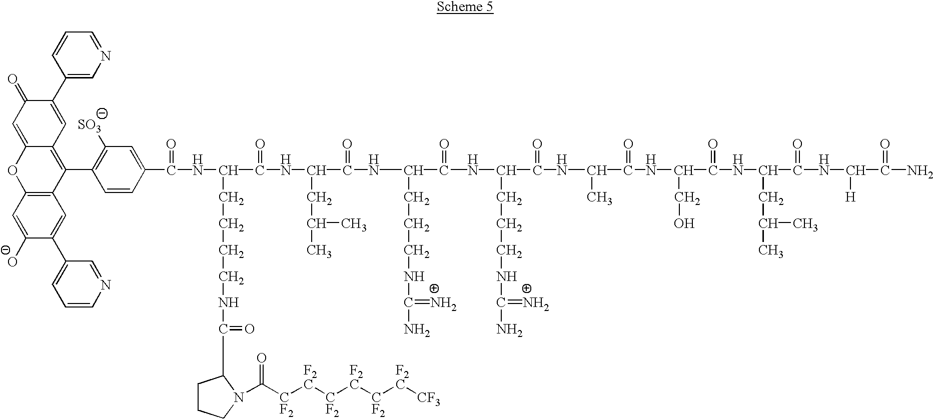

As can be seen, compound 8 contains a phenolate anion and a sulfonate anion in the Dye moiety for a total negative charge of −2. This negative charge on the dye is offset by the two positively charged guanidinium groups in the two arginine residues, for a total of two positive charges. Thus, the net charge of compound 8 is about 0 at pH 8.

Compound 8 further includes a protein kinase recognition moiety in the form of a polypeptide containing an amino acid sequence that is recognized by protein kinase A. The recognition moiety also contains an unphosphorylated serine that is capable of being phosphorylated by the kinase. Upon phosphorylation, the net charge of the substrate is changed from neutral to −2, thereby causing an increase in fluorescence. Fluorescence data for compound 8 can be found in Example 5B.

In still another embodiment, the present invention encompasses enzyme substrates that include a further spacer. Scheme 6 shows an exemplary substrate (compound 9) for detecting protein kinase A activity that is contemplated by this embodiment. The compound can be represented as N-Ac-ArgGlyArgProArgThrSerSerPheAlaGluGly-OOOLys(ε-N-Dye)Lys(ε-N—X)—NH₂, wherein X is an octadecanoyl group that is linked to the epsilon amino group of a lysine residue, Dye is a fluorescent moiety (5-carboxy-sulfofluorescein) that is linked to the epsilon amino group of a lysine residue, 0 is a linker provided from a 2-aminoethoxy-2-ethoxyacetyl group, and NH2 indicates that the carboxyl group of the C-terminal glycine is an amide. An exemplary synthesis of compound 9 is described in Example 6A. In this structure, the hydrophobic X group is linked to the epsilon amino group of a lysine residue without any further linker atoms. However, it will be appreciated that a linker containing one or more linking chain atoms could also be included if desired. Furthermore, the fluorescent dye is linked directly by an amide bond to the epsilon amino group of a lysine residue, without using additional linker atoms. However, it will be appreciated that a linker containing one or more linking chain atoms could also be included if desired.

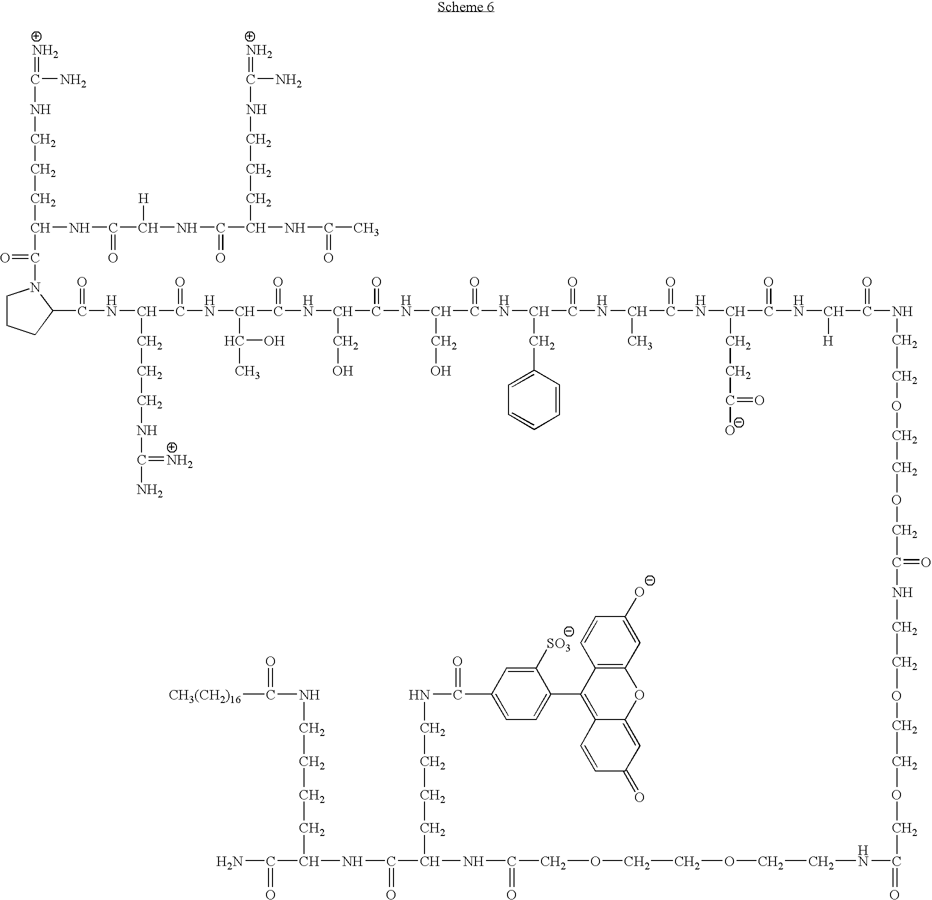

As can be seen, compound 9 contains a phenolate anion and a sulfonate anion in the Dye moiety and a carboxylate on the side chain of the glutamate residue, for a total negative charge of −3. This negative charge on the dye is offset by the three positively charged guanidinium groups in the three arginine residues, for a total of three positive charges. Thus, the net charge of compound 9 is about 0 at pH 8.

Compound 9 further includes a protein kinase recognition moiety in the form of a polypeptide containing an amino acid sequence that is recognized by protein kinase A. The recognition moiety also contains two unphosphorylated serine residues and an unphosphorylated threonine residue, at least one of which is capable of being phosphorylated by the kinase. Fluorescence data for compound 8 can be found in Example 6B.

Further examples of kinase substrates wherein a linker is incorporated are shown in Table 3.

TABLE 3

| | | RFUs at 10 uL | Conc | Fold |
|---|---|---|---|---|
| Kinase | Peptide | (initial→final) | (uM) | increase |
| PKA | C13-K(dye2)-LRRASLG-NH₂ | 1000→5000 | 8 | 5× |
| PKA | C13-OOOK(dye2)-LRRASLG-NH₂ | 1000→5000 | 8 | 5× |
| PKC | C16-OOOK(dye2)-RREGSFR-NH₂ | 650→3000 | 4 | 4.5× |
| PKC | C17-OOOK(tet)-RQGSFRA-NH₂ | 700→4900 | 6 | 7× |
| Src, lyn, fyn | C16-OOOK(dye2)RIGEGTYGVVRR-NH₂ | 1000→6500 | 8 | 6.5× |
| Akt | C15-OOOK(dye2)RPRTSSF-NH₂ | 1500→7500 | 8 | 4× |
| MAPK | C17-OOOK(dye2)PRTPGGR-NH₂ | 1100→5700 | 16 | 5× |
| MAPKAP2 | C16-OOOK(dye2)RLNRTLSV-NH₂ | 800→3200 | 8 | 4× |

In Table 3, each “O” represents a linker provided by a 2-aminoethoxy-2-ethoxyacetyl group; “dye 2” is a fluorescent moiety provided by 5-carboxy-2′,7′-dipyridyl-sulfonefluorescein; “tet” is a fluorescent moiety provided by 2′,7′,4,7-tetachloro-5-carboxy fluorescein (2′,7′-dichloro-5-carboxy-4,7-dichlorofluorescein); and NH2 indicates that the carboxy group of the C-terminal amino acid residue is amidated.

As can be seen from the data in Table 3, kinases of several classes exhibit similar increases in fluorescence under phosphorylation assay conditions. An exemplary synthesis of one exemplary member of these substrates (compound 10, Scheme 7) is described in Example 7.

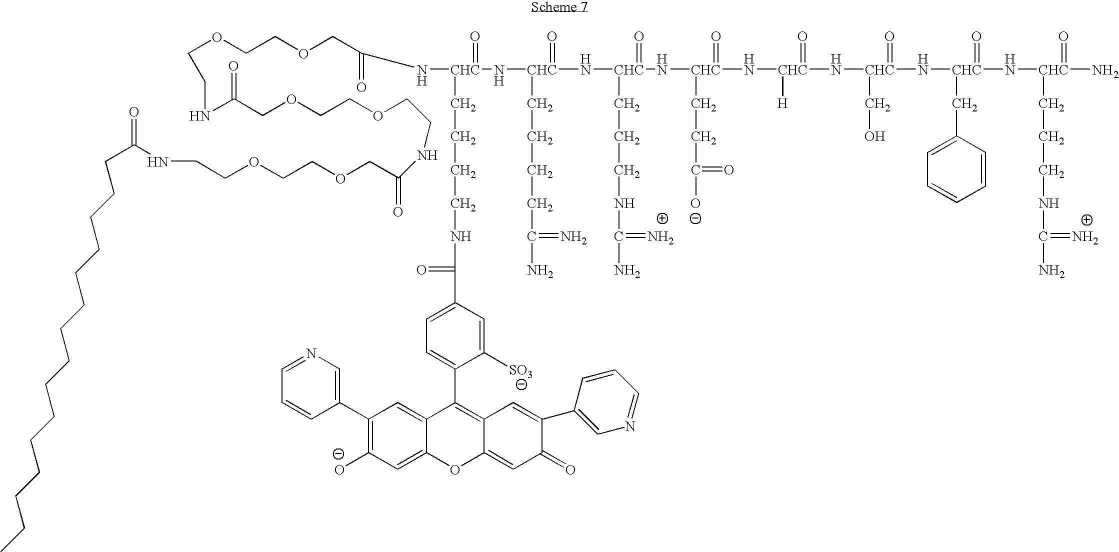

The compound can be represented as N—X—OOOLys(ε-N-Dye)ArgArgGluGly-SerPheArg-NH₂, wherein X is an hexadecanoyl group that is linked to the α-amino group of the lysine residue by the linker —OOO—, Dye is a fluorescent moiety (5-carboxy-2′,7′-dipyridyl-sulfofluorescein) that is linked to the epsilon amino group of the lysine residue, O is a linker provided from a 2-aminoethoxy-2-ethoxyacetyl group, and NH2 indicates that the carboxyl group of the C-terminal glycine is an amide. An exemplary synthesis of compound 10 is described in Example 7A. In this structure, the hydrophobic X group is linked to the α-amino group of the lysine residue by the linker —OOO—. It will be appreciated that alternative linkers could also be included if desired. Furthermore, the fluorescent dye is linked directly by an amide bond to the N-terminal amino group of the polypeptide segment, without using additional linker atoms. However, it will be appreciated that a linker containing one or more linking chain atoms could also be included if desired.

As can be seen, compound 10 contains a phenolate anion and a sulfonate anion in the Dye moiety and a carboxylate on the side chain of the glutamate residue, for a total negative charge of −3. This is offset by the guanidinium groups in the three arginine residues, for a total of three positive charges. Thus, the net charge of the compound is about 0 at pH 8.

In the various exemplary embodiments of kinase substrates illustrated herein, any terminal carboxyls are amidated. For example, in compounds 1, 2, 3, 4, 5, 6, 7, 8 and 10, the C-terminus of the kinase recognition moiety is amidated. In compound 9, the free C-terminus of the lysine residue linking the hydrophobic moiety is amidated. When amidated, such C-terminal carboxyls do not contribute to the net charge of the substrate at pH 8. It will be appreciated that other groups could be used to “mask” the charge contribution of terminal carboxyls (as well as side chain carboxyls, if desired). For example, such carboxyls could be esterified. Alternatively, such carboxyls can be unmasked and used to contribute to the overall net charge of the substrate.

The present invention contemplates not only detecting target enzymes, but also methods involving: (1) screening for and/or quantifying enzyme activity in a sample, (2) determining kcat and/or Km of an enzyme or enzyme mixture with respect to selected substrates, (3) detecting, screening for, and/or characterizing substrates of enzymes, (4) detecting, screening for, and/or characterizing inhibitors, activators, and/or modulators of enzyme activity, and (5) determining substrate specificities and/or substrate consensus sequences or substrate consensus structures for selected enzymes.

For example, in screening for enzyme activity, a sample that contains, or may contain, a particular enzyme activity is mixed with a substrate of the invention, and the fluorescence is measured to determine whether an increase in fluorescence has occurred. Screening may be performed on numerous samples simultaneously in a multi-well or multi-reaction plate or device to increase the rate of throughput. Kcat and Km may be determined by standard methods, as described, for exaample, in Fersht, Enzyme Structure and Mechanism, 2nd Edition, W. H. Freeman and Co., New York, (1985)).

In some embodiments, the reaction mixture may contain two or more different enzymes. This may be useful, for example, to screen multiple enzymes simultaneously to determine if at least one of the enzymes has a particular enzyme activity.

The substrate specificity of an enzyme can be determined by reacting an enzyme with different substrates having different enzyme recognition moieties, and the activity of the enzyme toward the substrates can be determined based on an increase in their fluorescence. For example, by reacting an enzyme with several different substrates having several different protein kinase recognition moieties, a consensus sequence for preferred substrates of a kinase can be prepared.

Each different substrate may be tested separately in different reaction mixtures, or two or more substrates may be present simultaneously in a reaction mixture. In embodiments in which the different substrates are present simultaneously in the reaction mixture, the substrates can contain the same fluorescent moiety, in which case the observed fluorescent signal is the sum of the signals from enzyme reaction with both substrates. Alternatively, the different substrates can contain different, fluorescently distinguishable fluorescent moieties that allow separate monitoring and/or detection of the reaction of enzyme with each different substrate simultaneously in the same mixture. The fluorescent moieties can be selected such that all or a subset of them are excitable by the same excitation source, or they may be excitable by different excitation sources. They can also be selected to have additional properties, such as, for example, the ability to quench one another when in close proximity thereto, by, for example, collisional quenching, FRET or another mechanism (or combination of mechanisms).

Although not necessary for operation of the methods, the assay mixture may optionally include one or more amphipathic quenching compounds designed to quench the fluorescence of the fluorescent moiety of the substrate (and/or plurality of substrates when more than one substrate is present in the mixture). Such amphipathic quenching molecules generally comprise a hydrophobic moiety capable of integrating the quenching compound into a micelle and a quenching moiety. The hydrophobic moiety can by any moiety capable of integrating the compound into a micelle, and as specific nonlimiting exemplary embodiments, can comprise any of the hydrophobic moieties described previously in connection with, for example, the kinase substrates.

The quenching moiety can include any moiety capable of quenching the fluorescence of the fluorescent moiety of the enzyme substrate used in the assay (or one or more of the substrates if a plurality of substrates are used). Compounds capable of quenching the fluorescence of the various different types of fluorescent dyes discussed above, such as xanthene, fluorescein, rhodamine, cyanine, pthalocyanine and squaraine dyes, are well-known. Such quenching compounds can be non-fluorescent (also referred to as “dark quenchers” or “black hole quenchers”) or, alternatively, they may themselves be fluorescent. Examples of suitable non-fluorescent dark quenchers that can comprise the quenching moiety include, but are not limited to, Dabcyl, the various non-fluorescent quenchers described in U.S. Pat. No. 6,080,868 (Lee et al.) and the various non-fluorescent quenchers described in WO 03/019145. (Ewing et al.). Examples of suitable fluorescent quenchers include, but are not limited to, the various fluorescent dyes described above in connection with kinase substrates.

The ability of a quencher to quench the fluorescence of a particular fluorescent moiety may depend upon a variety of different factors, such as the mechanisms of action by which the quenching occurs. The mechanism of the quenching is not critical to success, and may occur, for example, by collision, by FRET, by another mechanisms or combination of mechanisms. The selection of a quencher for a particular application can be readily determined empirically. As a specific example, the dark quencher Dabcyl and the fluorescent quencher TAMRA have been shown to effectively quench the fluorescence of a variety of different fluorophores. In a specific embodiment, a quencher can be selected based upon its spectral overlap properties spectral overlap with the fluorescent moiety. For example, a quencher can be selected that has an absorbance spectrum that sufficiently overlaps the emission spectrum of the fluorescent moiety such that the quencher quenches the fluorescence of the fluorescent moiety are in close proximity to one another, such as when the quencher molecule and substrate including the quencher moiety are integrated into the same micelle.

In embodiments in which a plurality of substrates are present in the assay, such as the multiplexed embodiments described above, it may be desirable to select a quenching moiety that can quench the fluorescence of the fluorescent moieties of all of the substrates present in the assay.

The hydrophobic and quenching moieties can be connected in any way that permits them to perform their respective functions. As a specific example, the hydrophobic moiety may be linked directly to the quenching moiety without the aid of a linker. Non-limiting examples of such quenching compounds include molecules in which a dye (e.g. a rhodamine or fluorescein dye) which contains a primary amino group (or other suitable group) is acylated with a fatty acid. As another specific example, the linkage may be mediated by way of a linker. The identity of the linker is not critical, and can include a peptide segment (or analog thereof). Although in many embodiments the peptide segment will not include an enzyme recognition moiety recognized by the enzyme(s) being assayed, it may optionally include such a moiety(ies). As a specific example, the quencher molecule can be a derivative or analog of any of the kinase or other enzyme substrates described herein in which the fluorescent moiety is replaced with a quenching moiety and the sequence of the enzyme recognition moiety is modified such that it is not recognized by the enzyme(s) being assayed in the sample.

Like the enzyme substrate, the quencher molecule can be designed to have specified charge characteristics.

Detecting, screening for, and/or characterizing inhibitors, activators, and/or modulators of enzyme activity can be performed by forming reaction mixtures containing such known or potential inhibitors, activators, and/or modulators and determining the extent of increase or decrease (if any) in fluorescence signal relative to the signal that is observed without the inhibitor, activator, or modulator. Different amounts of these substances can be tested to determine parameters such as Ki (inhibition constand), K_{H} (Hill coefficient), Kd (dissociation constant) and the like to characterize the concentration dependence of the effect that such substances have on enzyme activity.

Example 8 describes an inhibition study with a protein kinase A (see also FIG. 4). PKA from E. coli was incubated in the presence of different concentrations of ATP (50, 10, 3 and 2 μM adenosine triphosphate) in the absence (lowest trace) or presence of the inhibitor staurosporine (5 nM, middle trace) or the PKA-specific inhibitor TYADFIASGRTGRRNAI (20 nM, highest trace). The fluorescent substrate for phosphorylation had the structure: N-pahnitoyl-alpha-2-aminomethyl-Gly(5-carboxy-sulfonefluorescein)LeuArgArgAlaSer(OH)LeuGly-NH₂ (compound 11), wherein a hydrophobic moiety (palmitoyl) and a fluorescent moiety (Dye) are both linked to the N-terminal residue of the kinase recognition moiety, similar to structure shown in Scheme 3 above. The Dye, 5-carboxy-sulfonefluorescein, is linked to the N-terminal residue by an amide bond formed between the 5-carbonyl group and the 2-amino nitrogen of the 2-aminomethyl group. The palmitoyl group is coupled to the N-terminal residue via the alpha amino nitrogen. The structure is shown in Scheme 8, and a synthetic procedure is provided in Example 8A.

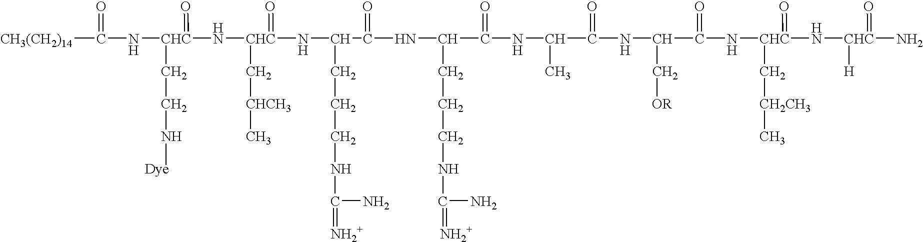

Results of the assay (Example 8B) are shown in FIG. 4 as a double reciprocal plot (1/V as a function of 1/S). As can be seen, when compared to the no-inhibitor control, the rate of enzyme-mediated phosphorylation was inhibited by both inhibitors. Since the plots for the inhibitors do not intersect with the y-intercept of the plot for the no-inhibitor control, neither inhibitor is a competitive inhibitor with respect to ATP binding (see for example A. Fersht, Enzyme Structure and Mechanism, 2nd Edition, W. H. Freeman and Co., New York, Chapter 3 (1985)). This result is consistent with the fact that these inhibitors bind to sites in the kinase that are different from the binding site for ATP.

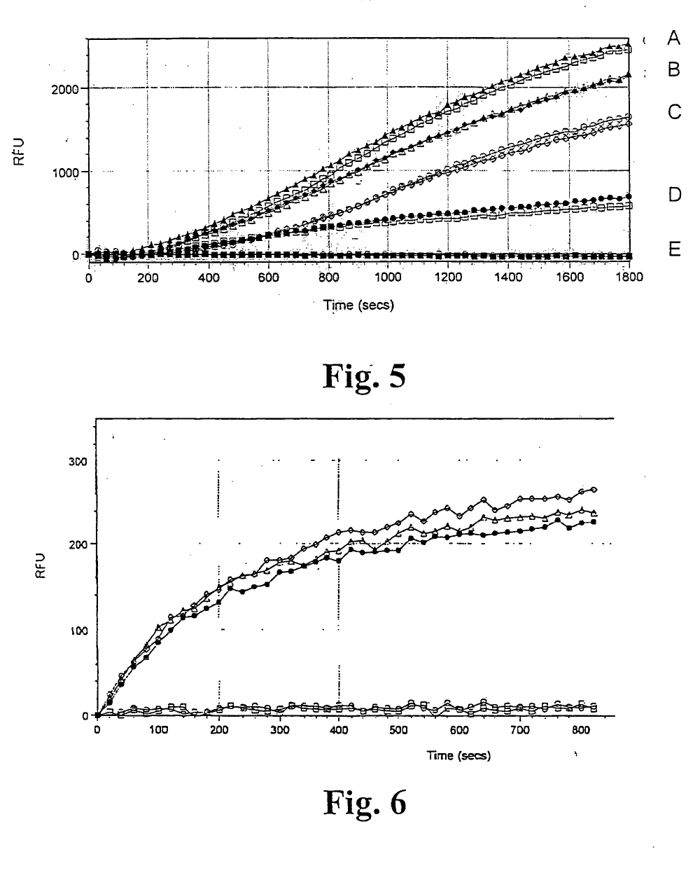

An inhibition study for protein kinase C-βII is described in Example 9. A PKC substrate was prepared (compound 12) having a general structure similar to that of the PKA substrate shown in Scheme 4 above, except that the protein kinase recognition moiety contained a peptide sequence designed for reaction with PKC-βII. In particular, the substrate contained a C-terminal tyrosine residue (Y) that can be phosphorylated by PKC-βII. Reaction mixtures were prepared in duplicate containing compound 12 in the presence of different amounts of the inhibitor, staurosporine (0, 2 nM, 5 nM, and 10 nM-traces A-D). A no-enzyme control was also performed (trace E). Results are shown in FIG. 5. As can be seen, kinase activity decreased with increasing inhibitor concentration.

Example 10 describes an assay for pp60^{c-src}-related protein tyrosine kinase. In this study, a substrate was prepared (compound 13) having a general structure similar to that of compound 12, except that the protein kinase recognition moiety contained a peptide sequence designed for the pp60^{c-src} kinase, and the site of phosphorylation is a tyrosine located internally within the recognition moiety. Enzyme reactions were performed in triplicate for a single substrate concentration, and two no-enzyme controls were also performed. Results of the assay are shown in FIG. 6. As can be seen, nearly identical fluorescence profiles were observed for the three identical substrate reactions, demonstrating the reproducibility and rapid fluorescent signal provided the assay conditions. Another noteworthy feature of this study is that despite the very small reaction volumes (5 μL) and low substrate concentration (2.5 μM), a strong fluorescent signal with high signal-to-noise was observed. This demonstrates the high sensitivity that can be obtained using the present invention.

Detection of fluorescent signal can be performed in any appropriate way. Advantageously, substrates of the invention can be used in a continuous monitoring phase, in real time, to allow the user to rapidly determine whether enzyme activity is present in the sample, and optionally, the amount or specific activity of the enzyme. The fluorescent signal is measured from at least two different time points, usually until an initial velocity (rate) can be determined. The signal can be monitored continuously or at several selected time points. Alternatively, the fluorescent signal can be measured in an end-point embodiment in which a signal is measured after a certain amount of time, and the signal is compared against a control signal (before start of the reaction), threshold signal, or standard curve.

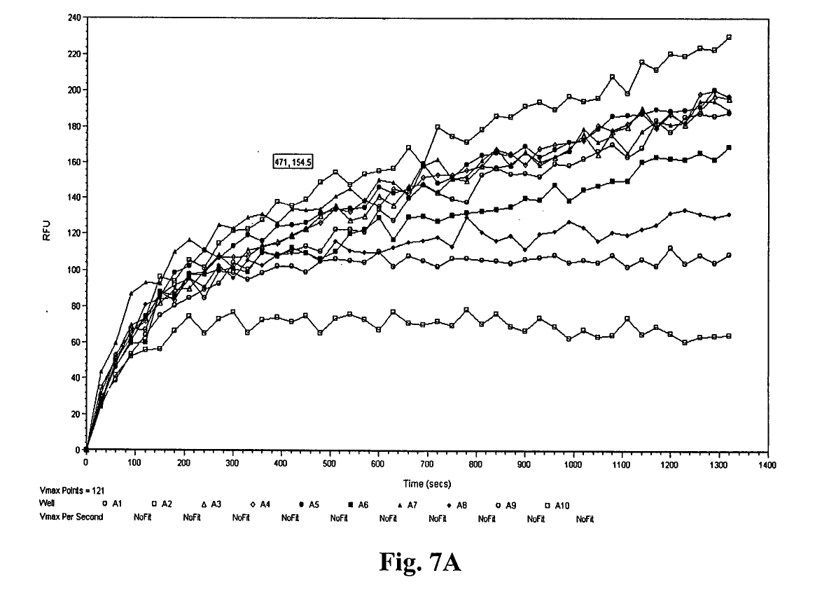

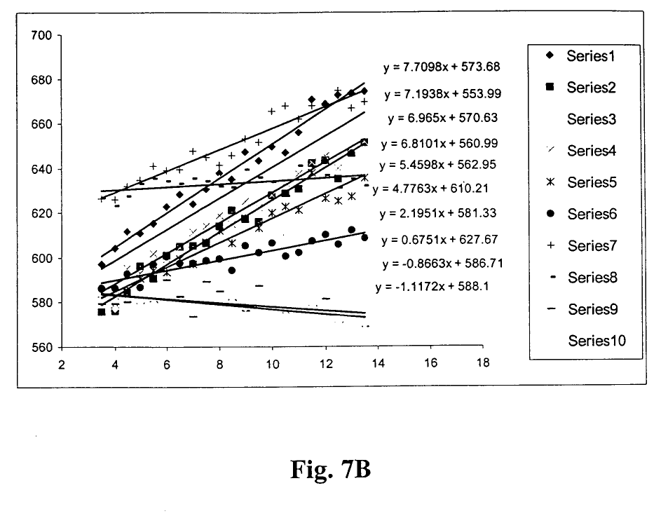

Example 11 describes a staurosporine-protein kinase C inhibition study to determine the IC₅₀ of staurosporine at various fixed ATP concentrations. Compound 10 from example 7, described above, was the substrate used for this study. The assays were conducted over a range of staurosporine concentrations (0, 0.1, 0.5, 1, 2, 5, 10, 20, 50 and 100 nM) at a fixed ATP concentration (either 10, 20, 50 or 100 μM) for each run. The raw kinetic data for 10 μM ATP are shown in FIG. 7A. The initial jump in fluorescence signal that is observed in FIG. 7A is due to the change in temperature that occurs when the reaction is allowed to warm from the incubation temperature (the temperature at which the reaction is held prior to the addition of ATP) of 0° C. to ambient temperature. As a result the fluorescence signal rises quickly over approximately the first 200 seconds. Because of this, the linear fitting shown in FIG. 7B was constructed from data taken between 3.5 to 13.5 minutes. The data in this range was used to calculate the slopes at different staurosporine concentrations, and the initial velocities were obtained from the slopes. Where the slope from the linear fit is negative, (for high staurosporine concentrations 50 and 100 nM respectively) a slope of 0 was applied.

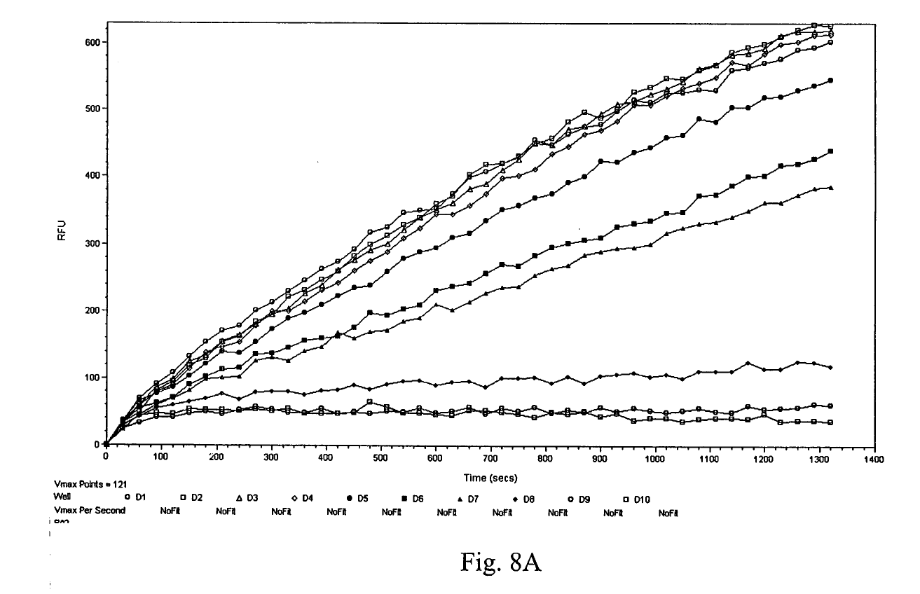

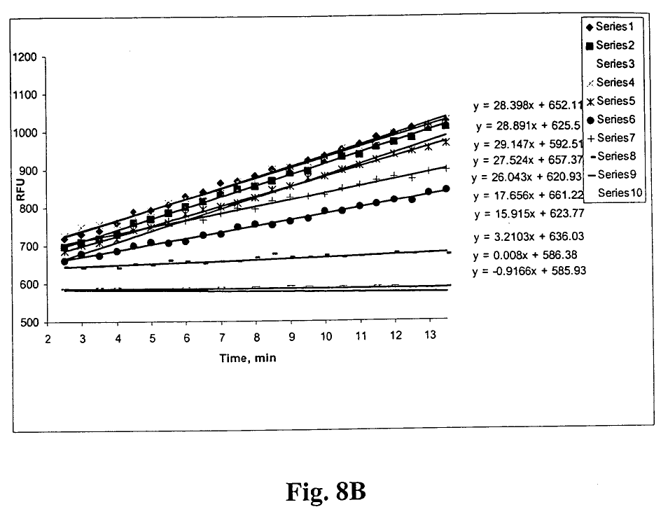

FIGS. 8A and 8B show raw kinetic data (FIG. 8A) and initial velocity data (FIG. 8B) for the same range of staurosporine concentrations run at 50 μM ATP. As can be seen in FIG. 8A, the signal to noise is improved over that of FIG. 7A. Also, the data used to do the linear fitting (FIG. 8B) was taken between 2.5 and 13.5 minutes in this case.

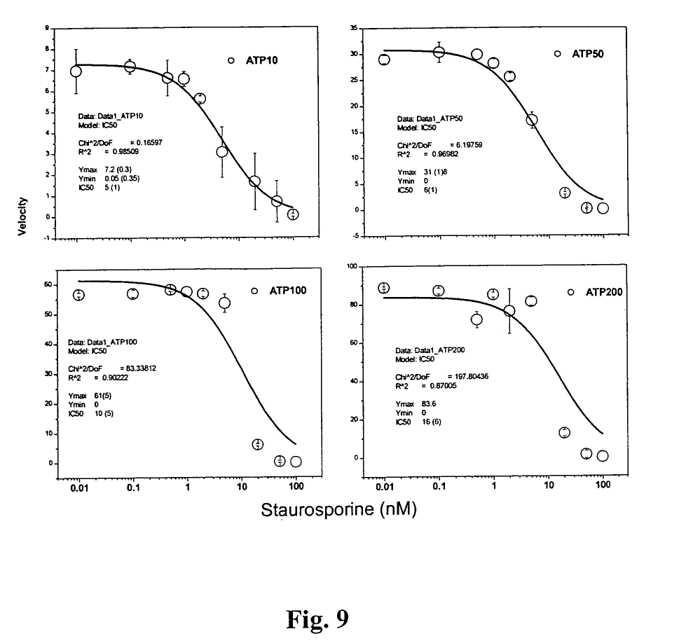

The IC₅₀s can be calculated from the initial velocities using methods known in the art. The IC₅₀ data are shown in FIG. 9. The IC₅₀s at ATP concentrations of 10, 50, 100 and 200 μM were found to be 5, 6, 10 and 16 nM respectively for this particular assay. These IC₅₀ values correspond well with those found in the literature.

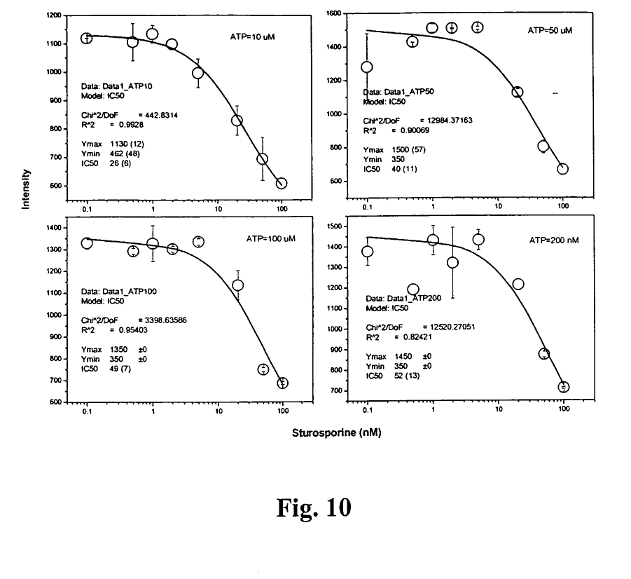

IC₅₀ values may alternatively be determined using an endpoint assay. FIG. 10 illustrates IC₅₀ values that were found using this method. The fluorescence intensity is measured after a one-hour reaction time. These values are generally 4 fold higher than the IC₅₀ values determined using initial velocities.

A comparison of the IC₅₀ values obtained by initial velocity and end point data respectively is shown in Table 4.

TABLE 4

| | | | | |
|---|---|---|---|---|
| ATP (uM) | 10 | 50 | 100 | 200 |
| IV (nM) | 5 | 6 | 10 | 16 |
| Endpoint (nM) | 26 | 40 | 49 | 52 |

IV. Kits

The invention also provides kits for performing methods of the invention. In one embodiment, the kit comprises at least one enzyme substrate for detecting a target enzyme, and a buffer for preparing a reaction mixture that facilitates the enzyme reaction. The buffer may be provided in a container in dry form or liquid form. The choice of a particular buffer may depend on various factors, such as the pH optimum for the enzyme to be detected, the solubility and fluorescence properties of the fluorescent moiety in the substrate, and the pH of the sample from which the target enzyme is obtained. The buffer is usually added to the reaction mixture in an amount sufficient to produce a particular pH in the mixture. In some embodiments, the buffer is provided as a stock solution having a pre-selected pH and buffer concentration. Upon mixture with the sample, the buffer produces a final pH that is suitable for the enzyme assay, as discussed above. The pH of the reaction mixture may also be titrated with acid or base to reach a final, desired pH. The kit may additionally include other components that are beneficial to enzyme activity, such as salts (e.g., KCl, NaCl, or NaOAc), metal salts (e.g., Ca2+ salts such as CaCl₂, MgCl₂, MnCl₂, ZnCl₂, or Zn(OAc), detergents (e.g., TWEEN 20), and/or other components that may be useful for a particular enzyme. These other components can be provided separately from each other or mixed together in dry or liquid form.

The enzyme substrate can also be provided in dry or liquid form, together with or separate from the buffer. To facilitate dissolution in the reaction mixture, the enzyme substrate can be provided in an aqueous solution, partially aqueous solution, or non-aqueous stock solution that is miscible with the other components of the reaction mixture. For example, in addition to water, a substrate solution may also contain a cosolvent such as dimethyl formamide, dimethylsulfonate, methanol or ethanol, typically in a range of 1%-10% (v:v).

For detection of protein kinase activity, the kit may also contain a phosphate-donating group, such as ATP, GTP, ITP (inosine triphosphate) or other nucleotide triphosphate or nucleotide triphosphate analog that can be used by the kinase to phosphorylate the substrate.

The operation of the invention can be further understood in light of the following non-limiting examples that illustrate various aspects of the invention.

### EXAMPLES

Materials and Methods

A. Reagents

Resins and reagents for peptide synthesis, Fmoc amino acids, 5-carboxyfluorescein succinimidyl ester were obtained from Applied Biosystems (Foster City, Calif.). Fmoc-Lys(Mtt)-OH, Fmoc-Ser(OPO(OBzl(OH)—OH and Fmoc-Dpr(ivDde) were obtained from Novabiochem. Protein kinase A and protein kinase C were obtained from Promega (Madison, Wis.). Staurosporine and ATP were obtained from Sigma-Aldrich (St. Louis, Mo.). Protein kinase C βII and src, active, were obtained from Upstate, Inc. (Lake Placid, N.Y.). All other chemicals and buffers were obtained from Sigma/Aldrich.

B. Instrumentation

Peptide synthesis was performed on an Applied Biosystems Model 433A Peptide Synthesizer. HPLC was performed on an Agilent 1100 series HPLC. UV-Vis measurements were performed on a Cary 3E UV-Vis spectrophotometer. Mass spectral data were obtained on a PE Sciex API 150EX mass spectrometer using electrospray ionization.

C. Absorbance Measurements

Concentrations of dye-labeled peptides were determined by dilution of the purified peptides into trifluoroethanol (500 μL) with 1 M pH 9 AMPSO (3-[(1,1-dimethyl-2-hydroxyethyl)amino]-2-hydroxypropanesulfonic acid) buffer (5 μL) and measuring the absorbance of the dye at its absorbance maximum (497 nm for 5-carboxyfluorescein). The extinction coefficient of 5-carboxyfluorescein was assumed to be 80,000 cm⁻¹M⁻¹.

D. Assay Reactions

Enzyme assay reactions were performed at ambient temperature. Protein kinase C assay reactions were carried out in 384 well, NBS, black plates (Corning, N.Y.).

### Example 1

### Protein Kinase Detection

A. Protein Kinase Substrate (Compound 1)

An exemplary enzyme substrate useful for detecting protein kinase A, palmitoyl-FAM-S(OPO₃²⁻)LRRRRFSK(Ac)G-amide, was prepared as follows. The peptide Fmoc-L(R(Pmc))₄FS(tBu)K(Mtt)G was constructed via solid phase peptide synthesis using standard FastMoc™ chemistry on 625 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 ml eppendorf tube and treated with 1 mL of 5% trifluoroacetic acid (TFA) in dichloromethane (DCM), giving a characteristic yellow trityl color. The resin was precipitated by the addition of 0.1 mL methanol and washed (3×1 mL dimethylformamide, DMF). Diisopropylethylamine (50 μL) and capping solution (1 mL of a solution of acetic anhydride (0.5 M) and hydroxybenzotriazole (0.015. M) in N-methylpyrrolidone (NMP)) were added to the resin and the mixture was agitated for 10 minutes. The resin was washed (3×1 mL DMF) and treated with piperidine (1 mL of 20% piperdine in DMF). After 4 minutes, the resin was washed with DMF (6×1 mL). The resin was treated with Fmoc-Ser(OPO(OBzl)OH)—OH (10 mg), coupling solution (50 μL of a solution of HBTU (2-(1H-benzotriazol-1-yl)1,1,3,3-tetramethyluronium hexafluorophosphate, 0.45. M) and HOBT (1-hydroxybenzotriazole, 0.45 M) and diisopropylethylamine (20 μL). After agitation for 35 minutes, the resin was washed with DMF (3×1 mL) and treated with piperidine (1 mL of 20% piperidine in DMF). After 5 minutes the resin was washed with DMF (6×1 mL) and treated with 4′-(para-(Fmoc-NHCH₂)C₆H₄C(═O)NHCH₂)-5-FAM succinimidyl ester (10 mg) and diisopropylethylamine (35 uL). After 1 h of agitation the resin was washed (6×1 mL DMF) and treated with piperidine (20% piperidine in NMP). After 5 minutes the resin was washed with NMP (6×1 mL) and treated with palmitoyl chloride (5 μL) and diisopropylethylamine (35 μL). After 16 minutes of agitation the resin was washed (3×1 mL NMP, 1×1 mL 1:1 methanol/DCM), and dried in a vacuum centrifuge. The peptide was cleaved from the resin with 1 mL cleavage solution (950 μL TFA, 50 μL water, 25 μL triisopropylsilane, and 25 μL thioanisole). After 1.5 to 2 h the mixture was filtered and the filtrate concentrated to dryness on a rotary evaporator. The residue was dissolved in water (0.5 mL) and a portion purified by reverse-phase HPLC (Metachem Technologies column: 150×4.6 mm, Polaris C18, 5 μm) using a 10% to 40% gradient over 10 min of 0.1% TFA in acetonitrile vs. 0.1% TFA in water. The dye-labeled peptide was analyzed by ESI mass spectrometry, which resulted in the expected M/z=2141.

B. Detection of Protein Kinase Activity

Reaction mixtures (100 μL) were prepared containing 20 mM Tris buffer, pH 8.5, Mg-ATP (1 mM), cAMP. (1 μM), and different concentrations of compound 1 (0.15 μM, 0.3 μM, and 0.6 μM), to which the catalytic subunit of protein kinase A (2 units) was then added. Fluorescence was monitored on a Perkin-Elmer LS-50B luminescence spectrometer with 500 nm excitation and emission at 530 nm, and slit width at 5 nm. The initial velocities were plotted as a double reciprocal plot to provide a value of 0.3 μM for the Km of compound 1. Results are shown in FIGS. 1 and 2.

### Example 2

### Phosphatase Detection

A. Phosphatase Substrate (Compound 2)

Synthesis of an exemplary dye-labeled peptide, compound 2, palmitoyl-LRRRRFS(OPO₃²⁻)K(5-FAM)G-amide, is described below. The peptide Fmoc-LR(Pmc)₄FS(OPO(OBzl)OH)K(Mtt)G was constructed via solid phase peptide synthesis using standard FastMoc™ chemistry on 625 mg of Fmoc-PAL-PEG-PS resin at 0.16 mmol/g, a solid support which results in a carboxamide peptide. Peptides with a carboxy terminus were constructed using Fmoc-Gly-PEG-PS resin. A portion of the final protected peptide-resin (20 mg, 2 μmol peptide) was transferred to a 1.5 ml eppendorf tube and treated with 1 mL of 5% trifluoroacetic acid (TFA) in dichloromethane (DCM), giving a characteristic yellow trityl color. The resin was precipitated by the addition of 0.1 mL methanol and washed (3×1 mL dimethylformamide, DMF). 5-Carboxyfluorescein succinimidyl ester (5 mg, 10 μmol), diisopropylethylamine (30 μL, 173 μmol) and DMF (100 μL) were added to the resin and the mixture was agitated gently for 2-10 h. The resin was washed (5×1 mL DMF), treated 5 minutes with piperidine (1 mL of 20% piperidine in NMP). The resin was washed with NMP (3×1 mL NMP). Palmitoyl chloride (5 μL) and diisopropylethylamine (35 μL) were added to the resin and the mixture agitated for 10 minutes. The resin was washed (3×1 mL NMP, 1×1 mL 1:1 methanol/DCM), and dried in a vacuum centrifuge. The peptide was cleaved from the resin with 1 mL cleavage solution (950 μL TFA, 50 μL water, 25 μL triisopropylsilane, and 25 μL thioanisole). After 1.5 to 2 h the mixture was filtered and the filtrate concentrated to dryness on a rotary evaporator. The residue was dissolved in water (0.5 mL) and a portion purified by reverse-phase HPLC (Metachem Technologies column: 150×4.6 mm, Polaris C18, 5 um) using a 10% to 40% gradient over 10 min of 0.1% TFA in acetonitrile vs. 0.1% TFA in water. The dye-labeled peptide was analyzed by ESI mass spectrometry, which resulted in the expected M/z=1850.

B. Detection of Phosphatase Activity

Phosphatase reactions were performed in a 100 μL reaction mixture containing compound 2 (20 μM), Tris HCl buffer (50 mM) at pH 9, and MgCl₂ (1 mM). Reactions were initiated by adding 0.5 units of E. coli alkaline phosphatase in 1 to 5 μL. The fluorescence was monitored on a Perkin-Elmer LS-50B luminescence spectrometer with excitation at 480 nm and emission at 525 and slit widths at 5 nm. Results are shown in FIG. 3.

### Example 3

### Dynamic Range of Fluorescence

This example describes a study to determine the difference in fluorescence between the phosphorylated and unphosphorylated forms of protein kinase A substrates. The purpose was to determine the extent to which the compounds could provide an increase in fluorescence signal, with good signal-to-noise, upon phosphorylation of the unphosphorylated forms.

A. Candidate Substrate (Compounds 3, 3P, 4, 4P, 5, 5P, 6 and 6P)

A series of compounds (Scheme 3 above) having different length alkylacyl groups were prepared in both phosphorylated and unphosphorylated form, represented by the following formula: X-Y(Dye)LRRAS(OR)LG-NH₂, wherein X is a fatty acid acyl group of the form CH₃(CH₂)ₙC(═O)—, x is 0, 7, 10, or 14, Y is alphα-aminomethyl glycine, Dye is a 4,7-dichlorofluorescein dye attached to the 2-amine nitrogen atom of Y by a 5-carbonyl linkage to the pendant phenyl ring of the dye, and R is H or PO₃²⁻.

For compounds 3-6, the peptide Fmoc-L(R(Pmc))₂AS(tBu)LG was constructed via solid phase peptide synthesis using standard FastMoc™ chemistry on 625 mg of Fmoc-PAL-PEG-PS resin at 0.16 mmol/g, a solid support which results in a carboxamide peptide. A representative synthesis of compound 4 follows.

A portion of the final protected peptide-resin (20 mg, 2 μmol peptide) was transferred to a 1.5 ml eppendorf tube and treated with piperidine (1 mL of 20% piperidine in DMF). After five minutes, the resin was washed with DMF (6×1 mL) and treated with Fmoc-Dpr(ivDde) (20 mg), coupling solution (100 μL, see above for composition) and diisopropylethylamine (40 μL). After 20 minutes of agitation, the resin was washed (4×1 mL DMF) and treated with piperidine (1 mL 20% piperidine in NMP). The resin was washed (4×1 mL NMP) and treated with nonanoyl chloride (5 μL) and diisopropylethylamine (30 μL). After 20 minutes of agitation, the resin was washed (5×1 mL NMP) and treated with hydrazine (1 mL of 2% in DMF). After 5 minutes, the resin was washed (5×1 mL DMF) and treated with a 4,7-dichloro-5-carboxyfluorescein succinimidyl ester (4 mg) and diisopropylethylamine (30 μL). After 1 h the resin was washed (10×1 mL DMF, 1×1 mL 1:1 methanol:dichloromethane) and dried in a vacuum centriftige. The peptide was cleaved from the resin, purified and analyzed as described, above.

Compounds 3, 5, and 6 were prepared in the same manner, except substituting nonanyl chloride with either acetic anyhydride (compound 3), lauryl chloride (compound 5) or palmitoyl chloride (compound 6). Compounds 3P, 4P, 5P and 6P were made similarly to compounds 3-6, except that the peptide fmocLR(Pmc)₂AS(OPO(OBzl)OH)LG on PAL resin was used.

B. Dynamic Range of Fluorescence

Solutions of the different compounds were diluted to a final concentration of 5 μM in 100 mM Tris HCl buffer, pH 8.5. The concentrations of stock solutions were determined by diluting into trifluoroethanol and assuming an extinction coefficient of the dye of 80,000 cm⁻¹M⁻¹. Fluorescence was measured on a Perkin-Elmer LS-50B luminescence spectrometer with excitation at 500 nm and emission at 546 nm. The slit widths were either 5 for dilute solutions or 3 nm with an attenuation factor of 4.4 for the most highly fluorescent solutions. Results are shown in Table 2 above.

### Example 4

### Protein Kinase Detection (Compound 7)

A. Protein Kinase A Substrate (Compound 7)

The synthesis of an exemplary dye-labeled peptide, compound 7, N-(1H, 1H,2H,2H-Perfluorodecyl-1-thiol-2-acetyl)-K-(5-carboxysulfofluorescein) LRRASL-G-amide, is described below. The peptide Fmoc-K(ivDde)LRRASLG was constructed via solid phase peptide synthesis using standard FastMoc™ chemistry on 625 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 mL Eppendorf tube and treated with 20% piperidine (500 μL) in dimethylformamide (DMF) for 20 minutes to remove the FMOC protecting group. The resin was then washed with 3×1 mL of DMF followed by 3×1 ml of methylene chloride (DCM). Iodoacetic acid (2.5 mg, 13 μmol), 0.2 M Dicyclohexylcarbodiimide in ethyl acetate (EtOAc) (70 μl, 13 μmol), 0.2 M N-hydroxysuccinimide in EtOAc (200 μL, 40 μmol) and DMF (100 μL) were combined and allowed to stand for 30 minutes. This mixture was added to the resin and was agitated gently for 3 h. The resin was washed (5×1 mL DMF) followed by 5×1 ml DCM. 1H,1H,2H,2H Perfluorodecyl-1-thiol (35 mg, 75 μmol) in 100 μL DMF was added to the resin and was agitated gently for 15 hrs. The resin was washed (5×1 mL DMF) followed by 5×1 ml DCM. The ivDde protecting group was removed by treating the resin with 10% hydrazine (500 μL) in DMF for 20 minutes. The resin was washed with 3×1 mL DMF followed by 3×1 ml DCM. 5-carboxysulfofluorescein (5 mg, 10 μmol), 0.45 M HOBT/HBTU (40 μl, 18 μmol), 2 M diisopropylethylamine in NMP (20 μL, 40 μmol) and DMF (100 μL) were added to the resin and the mixture was agitated gently for 3 h. The resin was washed (5×1 mL DMF) followed by 5×1 ml DCM. The peptide was cleaved from the resin with 1 mL cleavage solution (950 μL TFA, 50 μL water, 25 μL triisopropylsilane, and 25 μL thioanisole). After 1.5 to 2 h the mixture was filtered and the filtrate concentrated to dryness on a rotary evaporator. The residue was triturated with 3×1 mL hexane washes and a portion purified by reverse-phase HPLC (Agilent column: 150×4.6 mm, 300 Extend, 5 μM) using a 25% to 70% gradient over 25 min of 0.1% TFA in acetonitrile vs. 0.1% TFA in water. The dye-labeled peptide was analyzed by ESI mass spectrometry, which resulted in the expected M/z=1814.

B. Detection of Protein Kinase Activity

Fluorescence data were collected on a Perkin-Elmer LS-50B luminescence spectrometer. The fluorescence at 480 nm excitation and 520 nm emission of a 100 μL solution containing compound 7 (1 μM), Tris buffer, pH 8.1 (20 mM), MgCl₂ (1 mM) and protein kinase A (35 units) was found to be 35 fluorescence units. ATP was added to a final concentration of 0.5 mM and the fluorescence monitored, reaching a maximum of 180 fluorescence units after 6 minutes, or a 5-fold increase.

### Example 5

### Protein Kinase Detection (Compound 8)

A. Protein Kinase A Substrate (Compound 8)

The synthesis of an exemplary kinase substrate, compound 8, (5-carboxy-2,7-dipyridyl-sulfofluorescein)-K-(N-perfluoro-octanoyl-proline)-LRRASLG-amide, follows. The peptide Fmoc-K(ivDde)LRRASLG was constructed via solid phase peptide synthesis using standard FastMoc™ chemistry on 625 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 mL Eppendorf tube and treated with 20% piperidine (500 μL) in dimethylformamide (DMF) for 20 minutes to remove the FMOC protecting group. The resin was then washed with 3×1 mL of DMF followed by 3×1 mL of methylene chloride (DCM). 5-carboxy-2,′7′-dipyridyl-sulfofluorescein (5 mg, 9 μmol), 0.45M HOBTIHBTU (40 μL,18 μmol), 2M diisopropylethylamine in NMP((20 μl, 40 μmol) and DMF (100 μL) were added to the resin and the mixture was agitated gently for 3 hrs. The resin was washed (5×1 mL DMF) followed by 5×1 mL DCM. The ivDde protecting group was removed by treating the resin with 10% hydrazine (500 μL) in DMF for 20 minutes. The resin was washed with 3×1 mL DMF followed by 3×1 mL DCM. (N-Perfluorooctanoyl L-proline (prepared by the method of Curran and Luo, JACS 1999, 121, 9069-9072; 25 mg, 49 μmol), 0.45M HOBT/HBTU (40 μL, 18 μmol) and 2 M DIPEA/NMP (20 μl, 40 μmol) were added to the resin and agitated gently for 1 Shrs. The resin was washed (5×1 mL DMF) followed by 5×1 ml DCM. The peptide was cleaved from the resin with 1 mL cleavage solution (950 μL TFA, 50 μL water, 25 μL triisopropylsilane, and 25 μL thioanisole). After 1.5 to 2 h the mixture was filtered and the filtrate concentrated to dryness on a rotary evaporator. The residue was triturated with 3×1 mL hexane washes and a portion purified by reverse-phase HPLC (Agilent column: 150×4.6 mm, 300 EXTEND, 5 μm) using a 25% to 70% gradient over 25 min of 0.1% TFA in acetonitrile vs. 0.1% TFA in water. The dye-labeled peptide was analyzed by ESI mass spectrometry, which resulted in the expected M/z=1940.

B. Detection of Protein Kinase Activity

Fluorescence data were collected on a Perkin-Elmer LS-50B luminescence spectrometer. The fluorescence at 520 nm excitation and 550 nm emission of a 100 μL solution containing compound 8 (1 μM), Tris buffer, pH 8.1 (20 mM), MgCl₂ (1 mM) and protein kinase A (35 units) was found to be 140 fluorescence units. ATP was added to a final concentration of 0.5 mM and the fluorescence monitored, reaching a maximum of 493 fluorescence units after 5 minutes, or a 3.5-fold increase.

### Example 6

### Protein Kinase Detection (Compound 9)

A. Protein Kinase A Substrate (Compound 9)

The synthesis of exemplary substrate, compound 9, N-AcetylRGRPRTSSFAEG-OOOK(N-5-Carboxysulfo-fluroescein)K(N-Octadecanoyl)-amide, where 0 is a linker provided from a 2-aminoethoxy-2-ethoxyacetyl group, is described below. The peptide N-AcetylRGRPRTSSFAEG(AEEA)₃K(ivDde)K(Mtt) was constructed via solid phase peptide synthesis using standard FastMoc™ chemistry on 625 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 ml Eppendorf tube and treated with 5% trifluoroacetic acid (TFA) in dimethylformamide (DMF) (4×200 μL with 5 minute wait per treatment) to remove the mtt protecting group. The resin was then washed with 3×1 mL of DMF followed by 3×1 mL of methylene chloride (DCM) and is treated with octadecanoic acid (25 mg, 88 μmol), 0.45 M HOBT/HBTU(100 μl, 45 μmol), 2 M DIPEA in NMP (40 μL, 80 μmol) and DMF (200 EL). The mixture was gently agitated for 2 hrs and the resin was washed with 3×1 mL of DMF followed by 3×1 mL of DCM. The resin was treated with 10% hydrazine in DMF (500 μL) for 20 minutes to remove the ivDde protecting group, followed by washing with 3×1 mL DMF and 3×1 ml DCM. 5-carboxy-sulfofluorescein (5 mg, 9 μmol), 0.45 M HOBT/HBTU (40 μL, 18 μmol), 2 M DIPEA in NMP (20 μL, 40 μmol) and DMF (100 μL) were added to the resin and the mixture was agitated gently for 3 h. The resin was washed with 5×1 mL DMF followed by 5×1 mL DCM. The peptide was cleaved from the resin with 1 mL cleavage solution (950 μL TFA, 50 μL water, 25 μL triisopropylsilane, and 25 μL thioanisole). After 1.5 to 2 hrs the mixture was filtered and the filtrate concentrated to dryness on a rotary evaporator. The residue was triturated with 3×1 mL hexane washes and a portion purified by reverse-phase HPLC (Agilent column: 150×4.6 mm, 300 EXTEND, 5 μM) using a 25% to 70% gradient over 25 min of 0.1% TFA in acetonitrile vs. 0.1% TFA in water. The dye-labeled peptide was analyzed by ESI mass spectrometry, which resulted in the expected M/z=2714.

B. Detection of Protein Kinase Activity

Fluorescence data were collected on a Perkin-Elmer LS-50B luminescence spectrometer. The fluorescence at 480 nm excitation and 520 nm emission of a 100 μL solution containing compound 9 (1 μM), Tris buffer, pH 8.1 (20 mM), MgCl₂ (1 mM) and ATP (0.5 mM) was found to be 50 fluorescence units. Protein kinase A (7 Units) was added and the fluorescence monitored, reaching a maximum of 420 fluorescence units after 3 minutes, or an 8-fold increase.

### Example 7

### Protein Kinase Detection (Compound 10)

A. Protein Kinase C Substrate (Compound 10)

Synthesis of (N-palmitoyl)-Lys(N-5-carboxy-2,′7′-dipyridyl-sulfofluorescein)-OOO-RREGSFR-amide, is described below. The abbreviation 0 describes a linker provided from a 2-aminoethoxy-2-ethoxyacetyl group. The peptide Fmoc-OOOK(ivDde)RREGSFR was constructed via solid phase peptide synthesis using standard FastMoc™ chemistry on 625 mg of Fmoc-PAL-PEG-PS resin at 0.16 mmol/g, a solid support which results in a carboxamide peptide. A portion of the final protected peptide-resin (20 mg, 2 μmol peptide) was transferred to a 1.5 mL Eppendorf tube and treated with 20% piperidine (500 μL) in dimethylformamide (DMF) for 20 minutes to remove the fmoc protecting group. The resin was washed with 3×1 mL DMF followed by 3×1 mL DCM. Palmitic acid (20 mg, 78 μmol), 0.45M HOBT/HBTU (100 μL, 45 μmol), 2M diisopropylethylamine/NMP (40 μL, 80 μmol) were added to the resin and agitated gently for 2 h followed by the above washes. The ivDde protecting group was removed by treating the resin with 10% hydrazine (500 μL) in DMF for 20 minutes. The resin was washed with 3×1 mL DMF followed by 3×1 mL DCM.

5-carboxy-2,′7′-dipyridyl-sulfofluorescein (5 mg, 9 μmol), 0.45M HOBT/HBTU (40 μL,18 μmol), 2M DIEPA in NMP((20 μl, 40 μmol) and DMF (100 μL) were added to the resin and the mixture was agitated gently for 3 hrs. The resin was washed (5×1 mL DMF) followed by 5×1 mL DCM. The resin was then washed with 20% piperidine (500 μL) in DMF followed by 5×1 mL DCM to remove dye related impurities. The peptide was cleaved from the resin with 1 mL cleavage solution (950 μL TFA, 50 μL water, 25 μL triisopropylsilane, and 25 μL thioanisole). After 1.5 to 2 h the mixture was filtered and the filtrate concentrated to dryness on a rotary evaporator. The residue was trituated with 3×1 mL hexane washes and a portion purified by reverse-phase HPLC (Agilent column: 150×4.6 mm, 300 EXTEND, 5 μm) using a 25% to 70% gradient over 25 min of 0.1% TFA in acetonitrile vs. 0.1% TFA in water. The dye-labeled peptide was analyzed by ESI mass spectrometry, which resulted in the expected M/z=2255.

B. Protein Kinase C Assay Protocol

Protein kinase C reaction mixture aliquots (9 μL each) containing 2 μL 5× buffer (composed of 100 mM Tris, pH 8.1+25 mM MgCl₂, and 0.5% v/v β-mercaptoethanol) 1 μL Upstate Lipid Activator, 0.05 μL Promega Protein Kinase C, 5.55 μL deionized water, and 0.4 μL substrate were added to wells of the 384 well plate. The reactions were initiated by the addition of 1 μL of Sigma-Aldrich ATP into each well. Data were collected on a Molecular Devices Gemini Plate reader (Molecular Devices, Sunnyvale, Calif.) set at an excitation of 500 nm and an emission of 550 nm. Results are shown in Table 3.

### Example 8

### Inhibition of Protein Kinase A

A. Protein Kinase A Substrate

A fluorescent PKA substrate (compound 11) having the structure, N-palmitoyl-alpha-(2-aminomethyl)glycine(5-carboxy-sulfonefluorescein)LeuArgArgAlaSer(OH)Leu-Gly-NH₂, was prepared by a method similar to that used to make compound 6 above, but with the following changes. Instead of palmitoyl chloride, palmitic acid (5 mg), coupling solution (100 μL) and diisopropylethylamine (30 μL) were used for addition of the hydrophobic moiety. Also, the reaction to attach the fluorescent dye involved the use of 5-carboxysulfonefluorescein (5 mg), coupling solution (50 μL) and diisopropylamine (20 μL). This resulted in attachment of the fluorescent dye to the alphα-amino group of the N-terminal alpha-(2-aminomethyl)glycine residue, and attachment of the 5-carboxy-sulfonefluorescein to 2-amino nitrogen of the 2-aminomethyl group of the same residue by amide linkage to the 5-carbonyl group of the dye.

B. Inhibition of Kinase Activity

Reaction mixtures (100 μL) were prepared containing 20 mM Tris-HCl, pH 8.1, 1 mM MgCl₂, 1 μM compound, and 3 units of protein kinase A. Reactions were initiated by addition of ATP to a final concentration of 50, 10, 3 and 2 μM. Fluorescence data were collected on a Perkin-Elmer LS-50B luminescence spectrometer at an excitation of 480 nm and emission of 520 nm. The assay was repeated in the presence of staurosporine (5 nM) or a PKA-specific peptide inhibitor (20 nM) TYADFIASGRTGRRNAI. Results are shown in FIG. 4.

### Example 9

### Inhibition of Protein Kinase C-εII

A. Protein Kinase C-εII Substrate

A PKC substrate was prepared having the following structure (compound 12): alpha-palmitoyl-Lys(ε-N-5-carboxy-sulfonefluorescein)S(OPO₃²⁻)KLKRQGSFKY-amide, wherein a palmitoyl group is linked to the alpha amino group of the N-terminal lysine residue by an amide linkage, and the fluorescein dye was linked to the epsilon nitrogen atom of the same lysine residue by an amide linkage to the 5-carboxy group of the dye. The synthetic procedure was similar for that of the PKA substrates described above, except that Fmoc-S(PO(OBzl)OH)KLKRQGSFKY was formed on PAL resin, and Fmoc-Dpr(ivDde) was replaced with Fmoc-Lys(ivDde).

B. Inhibition of PKC Activity

Reaction mixtures (100 μL) were prepared containing 20 mM Tris-HCl, pH 8.1, 1 mM MgATP, 10 μL Upstate Lipid Activator, 2.5 μM PKC-βII substrate (compound 8), and various amounts of the inhibitor staurosporine (0, 2, 5, and 10 nM). Reactions were initiated by the addition of 8 ng PKC-βII enzyme. Data were collected on a Molecular Devices Gemini Plate reader (Molecular Devices, Sunnyvale, Calif.) set at an excitation of 485 nm and emission of 520 nm in kinetic mode. Results are shown in FIG. 5.

### Example 10

### Detection of pp60^{c-src}-Related Protein Tyrosine Kinase

A. Protein Tyrosine Kinase Substrate

A substrate (compound 13) was prepared having the structure: N-palmitoyl-Lys(ε-N-5-carboxy-sulfonefluorescein)KVEKIGEGTYGVVKK-amide. The synthetic protocol was similar to that used for synthesis of compound 8, except that a peptide-resin Fmoc-Lys(ivDde)-KVEKIGEGTYGVVKK was used. Results are shown in FIG. 6.

B. Tyrosine Kinase Activity

Fluorescent signals were followed in a Molecular Devices Gemini plate reader (Molecular Devices, Sunnyvale, Calif.) set at 485/520 nm excitation/emission wavelengths in kinetic mode. Five wells of a Corning 384-well plate, black with a non-binding surface and low volume wells (cat. No. 3676) (Corning, Inc., Acton, Mass.) were used. Each well contained 5 μL of a solution containing compound 9 (2.5 μM), MgCl₂ (1 mM), Tris buffer, pH 8.1 (20 mM) and src, active (1 unit, cat. No. 14-326). The kinase reaction was initiated in three of the wells by the addition of 1.25 μL of ATP (200 μM) to a final concentration of 50 μM. Results are shown in FIG. 6.

### Example 11

### Protein Kinase C IC50 of Staurosporine

A. Protein Kinase C Substrate

Compound 10 described in Example 7 was the substrate used for this study.

B. Protein Kinase C Assay

The enzyme concentration is 0.15 ng/μl in 10 μL volume containing 20 mM Tris-2+HCl pH 8.1, 1 M Mg, 10% lipid activator with ATP concentration specified in FIG. 9 legends. The substrate used was compound 10 and the final concentration was 3 μM for all of the reactions. The fluorescence intensity was monitored at 520 nm by exciting the sample at 480 nm with 515 nm cutoff filter in the Molecular Device plate reader.

All publications and patent applications mentioned herein are hereby incorporated by reference as if each publication or patent application was specifically and individually indicated to be incorporated by reference.

Although the invention has been described with reference to certain illustrative embodiments and examples, it will be appreciated that various modifications and variations can be made without departing from the scope and spirit of the invention.

What is claimed is:

1. A substrate compound comprising a hydrophobic moiety capable of integrating the compound into a micelle, a fluorescent moiety and an enzyme recognition moiety.

2. The substrate compound of claim 1 which has a net neutral charge in aqueous solution at a pH of about pH 8.

3. The substrate compound of claim 1 in which the enzyme recognition moiety comprises a protein kinase recognition sequence including at least one unphosphorylated residue capable of being phosphorylated by a protein kinase.

4. The substrate compound of claim 3 in which the at least one unphosphorylated reside is tyrosine, serine or threonine.

5. The substrate compound of claim 3 in which the protein kinase recognition sequence is recognized by a TK kinase, an AGC kinase, a CAMK kinase, a CMGC kinase, an STE kinase, a TKL kinase, a CKI kinase or a kinase belonging to the group “other.”

6. The substrate compound of claim 3 in which the protein kinase recognition sequence is recognized by a protein kinase A, a protein kinase C, a Src kinase, a Lyn kinase, a Fyn kinase, an Akt kinse, a MAP kinase a MAPKAP2 kinase or a cAMP dependent kinase.

7. The substrate compound of claim 3 in which the protein kinase recognition sequence comprises a peptide sequence selected from the group consisting of:

| | |
|---|---|
| -R-R-X-S/T-Z-; | (SEQ ID NO:1) |
| -R-X-X-S/T-F-F-; | (SEQ ID NO:2) |
| -S/T-P-X-R/K-; | (SEQ ID NO:3) |
| -P-X-S/T-P-; | (SEQ ID NO:4) |
| -K-K-K-K-R-F-S-F-K-; | (SEQ ID NO:5) |
| -X-R-X-X-S-X-R-X-; | (SEQ ID NO:6) |
| -L-R-R-L-S-D-S-N-F-; | (SEQ ID NO:7) |
| -K-K-L-N-R-T-L-T-V-A-; | (SEQ ID NO:8) |
| -E-E-I-Y-E/G-X-F-; | (SEQ ID NO:9) |
| -E-I-Y-E-X-I/V-; | (SEQ ID NO:10) |
| -I-Y-M-F-F-F-; | (SEQ ID NO:11) |
| -Y-M-M-M-; | (SEQ ID NO:12) |
| -E-E-E-Y-F-; | (SEQ ID NO:13) |
| -L-R-R-A-S-L-G-; | (SEQ ID NO:14) |
| -R-Q-G-S-F-R-A-; | (SEQ ID NO:15) |
| -R-I-G-E-G-T-Y-G-V-V-R-R-; | (SEQ ID NO:16) |
| -R-P-R-T-S-S-F-; | (SEQ ID NO:17) |
| -P-R-T-P-G-G-R-; | (SEQ ID NO:18) |
| -R-L-N-R-T-L-S-V-; | (SEQ ID NO:19) |
| and | |

analogs and conservative mutants thereof, wherein X represents any residue and Z represents a hydrophobic residue.

8. The substrate compound of claim 3 which has a net neutral charge in aqueous solution at a pH of about pH 8.

9. The substrate compound of claim 3 which has the structure: 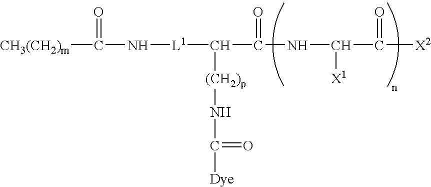
wherein:
m is an integer from 4 to 28;
n is an integer from 3 to 15;
p is an integer from 1 to 6;
L¹ is an optional linker;
Dye is a fluorescent dye which optionally includes a linker linking the Dye to the illustrated adjacent carbonyl group;
each X¹ is, independently of the others, an amino acid side chain; and
X² is OR or NH₂, where R is hydrogen or an alkyl containing from 1 to 8 carbon atoms,
with the proviso that the illustrated —[NH—CH(X¹)C(O)]ₙ—X² portion of the substrate compound includes at least one residue that is capable of being phosphorylated by a protein kinase.

10. The substrate compound of claim 9 in which L¹ is —[CH₂CH₂H₂CH₂O—CH₂C(O)NH]_{q}—, where q is 0, 1, 2 or 3.

11. The substrate compound of claim 9 in which Dye comprises a fluorescein or a rhodamine dye.

12. The substrate compound of claim 11 in which Dye comprises an optionally substituted structure selected from: 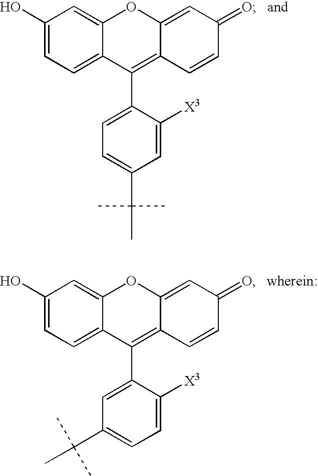
X³ is —C(O)O⁻ or —SO₃⁻ and the broken line indicates the point of attachment to the remainder of the illustrated structure.

13. The substrate compound of claim 12 in which Dye has the structure Dye2:

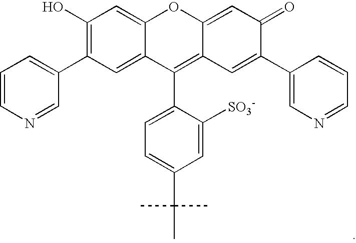

14. The substrate compound of claim 9 in which the illustrated —[NH—CH(X¹)C(O)]ₙ— portion of the substrate compound is a peptide is selected from the group consisting of:

| | |
|---|---|
| LRRASLG; | (SEQ ID NO:14) |
| RQGSFRA; | (SEQ ID NO:15) |
| RIGEGTYGVVRR; | (SEQ ID NO:16) |
| RPRTSSF; | (SEQ ID NO:17) |
| PRTPGGR; | (SEQ ID NO:18) |
| and | |
| RLNRTLSV. | (SEQ ID NO:19) |

15. The substrate compound of claim 3 in which the hydrophobic moiety comprises a substituted or unsubstituted, saturated or unsaturated hydrocarbon having from 6 to 30 carbon atoms.

16. The substrate compound of claim 15 in which the hydrocarbon is a linear, branched or cyclic, saturated or unsaturated alkyl.

17. The substrate compound of claim 16 in which the hydrocarbon is a linear alkyl containing from 10 to 26 carbon atoms.

18. The substrate compound of claim 17 in which the alkyl is fully saturated n-alkanyl.

19. The substrate compound of claim 17 in which the alkyl includes one or more carbon-carbon double bonds, each of which may, independently of the others, be in the cis or trans configuration and/or one or more carbon-carbon triple bonds.

20. The substrate compound of claim 3 in which the hydrophobic moiety contains at least one positively charged group.

21. The substrate compound of claim 3 in which the hydrophobic moiety contains at least one negatively charged group.

22. The substrate compound of claim 3 in which the fluorescent moiety comprises a dye selected from a xanthene dye, a rhodamine dye, a fluorescein dye, a cycanine dye, a phthalocyanine dye, a squaraine dye and a bodipy dye.

23. The substrate compound of claim 3 in which the fluorescent moiety comprises a fluorescence donor moiety and a fluorescence acceptor moiety.

24. The substrate compound of claim 23 in which the fluorescence donor moiety comprises a fluorescein dye.

25. The substrate compound of claim 23 in which the fluorescence acceptor moiety comprises a fluorescein or a rhodamine dye.

26. The substrate compound of claim 25 in which the fluorescence donor moiety comprises a fluorescein dye.

27. The substrate compound of claim 3 in which the fluorescent moiety comprises fewer than 150 atoms.

28. The substrate compound of claim 3 in which the hydrophobic moiety and the enzyme recognition moiety are linked to one another through the fluorescent moiety.

29. The substrate compound of claim 3 in which the hydrophobic moiety and the fluorescent moiety are linked to one another through the enzyme recognition moiety.

30. The substrate compound of claim 3 in which the hydrophobic moiety, the fluorescent moiety and the enzyme recognition moiety are linked to one another via a trivalent linker.

31. The substrate compound of claim 3 in which the hydrophobic moiety is linked to the fluorescent moiety by a linker than does not include a part of the enzyme recognition moiety.

32. The substrate compound of claim 3 in which the hydrophobic moiety is linked to the fluorescent moiety by a linker that includes at least a part of the enzyme recognition moiety.

33. The substrate compound of claim 1 in which the enzyme recognition moiety comprises a phosphatase recognition sequence including at least one phosphorylated residue capable of being dephosphorylated by a phosphatase.

34. The substrate compound of claim 33 which has a net neutral charge in aqueous solution at a pH of about pH 8.

35. A method of detecting the presence of an enzyme activity in a sample, comprising the steps of:
contacting the sample with a composition comprising a substrate compound according to claim 1 in which the enzyme recognition moiety is recognized by the enzyme, under conditions effective to permit the enzyme, when present in the sample, to modify the substrate compound in a manner that leads to an increase in a fluorescence signal produced by its fluorescent moiety; and
detecting a fluorescence signal, where an increase in the fluorescence signal indicates the presence and/or quantity of the enzyme in the sample.

36. The method of claim 35 in which the substrate compound is present at a concentration at or above its critical micelle concentration.

37. The method of claim 35 in which the fluorescence signal is detected as a function of time.

38. The method of claim 35 in which the composition further comprises a quenching compound which comprises a hydrophobic moiety capable of integrating the quenching compound into a micelle and a quenching moiety capable of quenching the fluorescence of the fluorescent moiety of the substrate compound.

39. The method of claim 35 which further comprises determining a Km value or Kcat value for an enzyme in the sample.

40. A method of identifying a compound that modulates an activity of an enzyme, comprising the steps of:
contacting the enzyme with a composition comprising a substrate compound according to claim 1 in which the enzyme recognition moiety is recognized by the enzyme in the presence of a candidate modulator compound and under conditions effective to permit the enzyme allow the enzyme to modify the substrate compound in a manner that leads to an increase in a fluorescence signal produced by its fluorescent moiety; and
detecting a fluorescence signal, where an increase or decrease in the fluorescence signal as compared to a control reaction or a standard curve indicates that the candidate modulator compound modulates the activity of the enzyme.

41. The method of claim 40 in which the candidate modulator compound is a known modulator of the enzyme activity and the method is used to assess the effect of the modulator compound on the activity of the enzyme.

42. The method of claim 40 in which is carried out to identify an inhibitor of the enzyme activity, where a decrease in the fluorescence signal as compared to a control reaction or a standard curve indicates that the candidate modulator compound inhibits the activity of the enzyme.

43. The method of claim 42 which further comprises determining the Ki of the inhibitor compound.

44. The method of claim 42 in which the candidate modulator compound is a known inhibitor of the activity of the enzyme and the method is used to determine the Ki of the compound.

45. A method of detecting phosphorylation activity of one or more protein kinases in a sample, comprising the steps of:
contacting the sample with a composition comprising a protein kinase substrate which comprises (1) a protein kinase recognition moiety containing at least one unphosphorylated residue capable of being phosphorylated by a protein kinase, (2) a hydrophobic moiety capable of integrating the substrate into a micelle, and (3) a fluorescent moiety, under conditions effective to allow phosphorylation of said residue when the protein kinase is present in the sample, thereby increasing a fluorescence signal produced by the fluorescent moiety; and
detecting a fluorescence signal, where an increase in the fluorescence signal indicates the presence and/or quantity of protein kinase phosphorylation activity in the sample.

46. The method of claim 45 in which the protein kinase substrate is a substrate compound according to any one of claims 3-32.

47. The method of claim 45 in which the fluorescence signal is detected as a function of time.

48. The method of claim 45 in which the composition further comprises a quenching compound which comprises a hydrophobic moiety capable of integrating the quenching compound into a micelle and a quenching moiety capable of quenching the fluorescence of the fluorescent moiety of the protein kinase substrate.

49. The method of claim 45 which further comprises determining a Km value or Kcat value for a protein kinase in the sample.

50. A method of identifying a compound that modulates phosphorylation activity of a protein kinase, comprising the steps of:
contacting the protein kinase with a composition comprising a protein kinase substrate which comprises (1) a protein kinase recognition moiety containing at least one unphosphorylated residue capable of being phosphorylated by a protein kinase, (2) a hydrophobic moiety capable of integrating the substrate into a micelle, and (3) a fluorescent moiety, in the presence of a candidate compound and under conditions effective to allow phosphorylation of said residue by the protein kinase, thereby increasing a fluorescence signal produced by the fluorescent moiety; and
detecting a fluorescence signal, where an increase or decrease in the fluorescence signal as compared to a control reaction or a standard curve indicates that the candidate compound modulates the activity of the protein kinase.

51. The method of claim 50 in which the candidate compound is a known modulator of the protein kinase phosphorylation activity and the method is used to assess the effect of the compound on the phosphorylation activity of the protein kinase.

52. The method of claim 50 in which is carried out to identify an inhibitor of the protein kinase phosphorylation activity, where a decrease in the fluorescence signal as compared to a control reaction or a standard curve indicates that the candidate compound inhibits the phosphorylation activity of the protein kinase.

53. The method of claim 50 which further comprises determining the Ki of the inhibitor compound.

54. The method of claim 50 in which the candidate compound is a known inhibitor of the activity of phosphorylation activity the protein kinase and the method is used to determine the Ki of the compound.

55. The method of claim 50 in which the composition further comprises a quenching compound which comprises a hydrophobic moiety capable of integrating the quenching compound into a micelle and a quenching moiety capable of quenching the fluorescence of the fluorescent moiety of the protein kinase substrate.


SEQUENCE LISTING (19 sequences)

SEQ ID NO: 1 - protein, 5 aa, Artificial
  Synthetic consensus sequence
RRXXX

SEQ ID NO: 2 - protein, 6 aa, Artificial
  Synthetic consensus sequence
RXXXFF

SEQ ID NO: 3 - protein, 4 aa, Artificial
  Synthetic consensus sequence
XPXX

SEQ ID NO: 4 - protein, 4 aa, Artificial
  Synthetic consensus sequence
PXXP

SEQ ID NO: 5 - protein, 9 aa, Artificial
  Synthetic consensus sequence
KKKKRFSFK

SEQ ID NO: 6 - protein, 8 aa, Artificial
  Synthetic consensus sequence
XRXXSXRX

SEQ ID NO: 7 - protein, 9 aa, Artificial
  Synthetic consensus sequence
LRRLSDSNF

SEQ ID NO: 8 - protein, 10 aa, Artificial
  Synthetic consensus sequence
KKLNRTLTVA

SEQ ID NO: 9 - protein, 7 aa, Artificial
  Synthetic consensus sequence
EEIYXXF

SEQ ID NO: 10 - protein, 6 aa, Artificial
  Synthetic consensus sequence
EIYEXX

SEQ ID NO: 11 - protein, 6 aa, Artificial
  Synthetic consensus sequence
IYMFFF

SEQ ID NO: 12 - protein, 4 aa, Artificial
  Synthetic consensus sequence
YMMM

SEQ ID NO: 13 - protein, 5 aa, Artificial
  Synthetic artificial sequence
EEEYF

SEQ ID NO: 14 - protein, 7 aa, Artificial
  Synthetic consensus sequence
LRRASLG

SEQ ID NO: 15 - protein, 7 aa, Artificial
  Synthetic consensus sequence
RQGSFRA

SEQ ID NO: 16 - protein, 12 aa, Artificial
  Synthetic consensus sequence
RIGEGTYGVVRR

SEQ ID NO: 17 - protein, 7 aa, Artificial
  Synthetic consensus sequence
RPRTSSF

SEQ ID NO: 18 - protein, 7 aa, Artificial
  Synthetic consensus sequence
PRTPGGR

SEQ ID NO: 19 - protein, 8 aa, Artificial
  Synthetic consensus sequence
RLNRTLSV